(19) United States
(12) Patent Application Publication
(10) Pub. No.: US 2026/0157871 A1
(43) Pub. Date: Jun. 11, 2026

(54) SMART KNEE ORTHOSIS WITH INTEGRATED BIOFEEDBACK FOR REAL-TIME PROPRIOCEPTIVE TRAINING

(71) Applicants: Khalid A. ALAHMARI, Abha (SA); Venkata Nagaraj KAKARAPARTHI, Abha (SA); Hani Hassan Hani Hassan, Abha (SA); Khalid A. ALSHAMRANI, Abha (SA); Paul Silvian SAMUEL, Abha (SA); Ravi Shankar REDDY, Abha (SA)

(72) Inventors: Khalid A. ALAHMARI, Abha (SA); Venkata Nagaraj KAKARAPARTHI, Abha (SA); Hani Hassan Hani Hassan, Abha (SA); Khalid A. ALSHAMRANI, Abha (SA); Paul Silvian SAMUEL, Abha (SA); Ravi Shankar REDDY, Abha (SA)

(21) Appl. No.: 19/337,067

(22) Filed: Sep. 23, 2025

(51) Int. Cl. A61F 5/01; A61B 5/296; A61B 5/375

(52) U.S. Cl. CPC A61F 5/0123; A61B 5/296; A61B 5/375

(57) ABSTRACT

A smart knee orthosis system provides structural stabilization of the knee joint and integrated proprioceptive training through real-time biofeedback. The orthosis includes an upper thigh frame and a lower shank frame interconnected by a hinge aligned with the anatomical knee axis. The hinge incorporates an encoder for continuous monitoring of angular displacement. Embedded within the orthotic structure are multi-modal sensors including inertial measurement units, strain gauges, and surface electromyography electrodes, which collectively capture joint kinematics, loading patterns, and neuromuscular activation. A microcontroller unit processes the acquired data using sensor fusion and adaptive control techniques to detect deviations from predetermined therapeutic thresholds. Based on the analysis, a biofeedback subsystem consisting of vibrotactile actuators, auditory transducers, and optional light indicators delivers immediate corrective cues to the wearer, thereby reinforcing proprioceptive awareness and neuromuscular retraining during functional activities. A wireless communication module enables data logging and remote clinical monitoring.

### TECHNICAL FIELD

The present invention relates to medical assistive and rehabilitative devices. More specifically, it pertains to a smart orthopedic knee orthosis integrated with embedded sensors, actuators, and biofeedback modules, configured to provide real-time proprioceptive training through active monitoring, feedback, and control of knee joint movements during therapeutic and daily activities.

### BACKGROUND

Conventional knee orthoses are widely used in clinical rehabilitation, sports injury recovery, and orthopedic therapy for stabilization and support of the knee joint. Traditional designs are largely passive in nature, providing mechanical restriction of movement or external bracing without dynamic interaction with the patient. While such devices prevent further damage and aid recovery, they lack the capability to engage the neuromuscular system actively through sensory feedback, which is essential for proprioceptive retraining.

Existing proprioceptive training protocols rely heavily on therapist-guided exercises, visual cues, or balance systems that require clinical supervision and specialized equipment. These methods are often resource-intensive, lack portability, and do not provide real-time adaptive correction during movement. There remains a technical gap in wearable rehabilitation devices that combine mechanical stabilization with sensor-driven feedback mechanisms capable of delivering proprioceptive cues during real-world functional movements.

The present invention addresses this unmet need by providing a smart knee orthosis equipped with embedded sensing arrays, real-time data acquisition, and actuator-driven biofeedback subsystems. The system transforms the orthosis from a passive brace into an intelligent rehabilitation device that continuously interacts with the wearer, detects deviations in joint motion, and provides corrective cues for proprioceptive engagement.

The field of orthotic and rehabilitative devices has long been driven by the need to stabilize, protect, and retrain joints after injury, surgery, or in cases of neuromuscular impairment. The knee joint, being one of the most complex and load-bearing structures of the human body, is highly susceptible to injury and degenerative conditions. Orthoses designed for the knee are traditionally aimed at providing external support and restricting motion within therapeutic limits. The earliest forms of knee braces were purely mechanical in nature, consisting of rigid or semi-rigid frames connected by mechanical hinges. Their purpose was to immobilize or limit the range of motion of the joint to protect damaged ligaments, cartilage, or soft tissues. Although these devices were structurally effective in restricting harmful motion, they did not address the underlying problem of proprioceptive loss, which is common after ligament injuries such as anterior cruciate ligament (ACL) tears or following surgical interventions. The passive nature of these devices left rehabilitation largely dependent on external physiotherapy and patient adherence to exercise programs.

Over time, improvements were made in terms of material science and ergonomics. Modern passive braces became lighter, more comfortable, and provided adjustable levels of restriction. Semi-rigid braces incorporated flexible materials to allow controlled motion, and unloader braces were developed to redistribute forces in cases of osteoarthritis. While these designs improved user comfort and compliance, they still operated as static support structures. Their effectiveness was primarily mechanical, offering no active interaction with the patient. As a result, proprioceptive retraining, which is crucial for restoring neuromuscular coordination and preventing recurrent injuries, remained dependent on separate therapy methods. This separation created inefficiencies in the rehabilitation process, requiring patients to spend extended time in clinics under therapist supervision.

To address these gaps, research efforts began integrating sensor technology into orthotic devices. The introduction of inertial measurement units (IMUs), strain gauges, and pressure sensors allowed orthoses to monitor joint motion and loading patterns. These sensor-augmented braces could provide clinicians with data regarding the range of motion, step counts, or weight-bearing symmetry. Commercially available smart braces and wearable motion trackers emerged, capable of logging activity data and transmitting it wirelessly to mobile applications. While these devices introduced a layer of digital interaction, they were largely limited to passive monitoring functions. The data collected was typically reviewed retrospectively by clinicians, meaning that real-time corrective action or proprioceptive training was still absent. Patients continued to rely on visual or verbal feedback from therapists rather than immediate sensory cues that could guide them during movement.

Another parallel development in rehabilitation technology involved the use of balance boards, virtual reality systems, and robotic exoskeletons. Balance boards with integrated sensors provided platforms for patients to perform stability exercises while tracking center-of-mass displacement. Virtual reality rehabilitation systems combined motion capture with immersive environments, engaging patients in interactive tasks designed to retrain balance and proprioception. Robotic exoskeletons offered high levels of control and feedback, assisting in gait training and repetitive motion therapy. Despite their technological sophistication, these solutions presented drawbacks in terms of cost, accessibility, and portability. Robotic exoskeletons and virtual reality setups were typically confined to clinical environments, requiring significant infrastructure and professional oversight. Patients could not feasibly use such systems in daily life, limiting the continuity of rehabilitation. Moreover, the complexity of these systems often made them intimidating for patients, reducing long-term compliance outside structured therapy sessions.

The growing field of biofeedback-based rehabilitation sought to bridge the gap between sensor-based monitoring and active engagement of the patient. Biofeedback methods employed cues such as visual displays, auditory signals, or tactile vibrations to inform patients about their performance during exercises. For instance, surface electromyography (sEMG) biofeedback systems allowed patients to observe their muscle activation patterns on a screen, training them to voluntarily engage or relax specific muscles. Similarly, auditory tones or haptic vibrations were used to indicate correct or incorrect joint positions. While biofeedback demonstrated clinical effectiveness in enhancing motor learning, the equipment was often standalone and not integrated into wearable orthotic devices. Patients needed to be connected to specialized sensors and displays during therapy sessions, again restricting the scope of use to clinics or controlled environments. This lack of integration into everyday wearable devices hindered the translation of biofeedback's benefits into continuous rehabilitation.

Another drawback of existing biofeedback systems lies in their dependence on visual or auditory channels, which can be intrusive or impractical during daily activities. Visual feedback requires constant attention to screens or indicators, which is not feasible during tasks such as walking or climbing stairs. Auditory cues, while more practical, may become socially disruptive or difficult to interpret in noisy environments. Tactile biofeedback, though promising, has not been widely integrated into commercial orthotic designs, leaving a gap in user-friendly proprioceptive engagement. Thus, while the theoretical benefits of biofeedback are well established, practical implementation in knee orthoses remains inadequate.

Recent advancements have also explored wearable technologies that integrate smartphones and fitness trackers for rehabilitation purposes. Smartphone applications can record accelerometer data, guide exercise routines, and provide reminders for therapy adherence. Fitness trackers equipped with motion sensors and haptic feedback can be adapted for monitoring knee activity. However, these solutions lack the structural support provided by orthoses and do not ensure accurate alignment with the anatomical knee joint. As a result, while they contribute to general activity tracking, they are insufficient for patients requiring both mechanical stabilization and proprioceptive retraining. The separation of mechanical orthoses and digital tracking devices creates a fragmented rehabilitation ecosystem, where patients must simultaneously use multiple devices, each addressing only part of the therapeutic need.

Another limitation of current orthotic devices and digital rehabilitation tools is their inability to personalize therapy dynamically. Most braces and tracking systems operate on pre-set configurations that do not adapt to the changing needs of a patient over the course of recovery. Rehabilitation is inherently dynamic: in the early stages, greater support and cautious feedback are necessary, while later stages demand increased challenge and active correction. Existing devices either remain rigidly supportive or overly simplistic in their feedback mechanisms. This lack of adaptability can slow recovery, reduce patient motivation, and fail to prevent secondary injuries arising from improper movements during unsupervised sessions.

From a clinical standpoint, there is also a challenge in maintaining accurate long-term data records for assessing patient progress. While some smart braces and wearables transmit data to external platforms, the granularity of this data is often limited. Many systems record only step counts or general motion ranges without detailed kinematic or neuromuscular information. This restricts the ability of clinicians to fine-tune therapy protocols and assess proprioceptive improvements objectively. The absence of integrated sEMG monitoring in most commercial orthoses further limits insights into muscle activation patterns, which are crucial for comprehensive rehabilitation.

In addition, issues of user compliance remain a significant barrier. Patients are more likely to adhere to rehabilitation protocols if the devices are comfortable, lightweight, and unobtrusive in daily life. Many sensor-augmented braces are bulky, require frequent charging, or involve complicated setup procedures, discouraging consistent use. Moreover, patients often fail to recognize the importance of proprioceptive training compared to mechanical support, leading to underutilization of devices that do not actively engage them with feedback. Without immediate sensory cues reinforcing correct motion, patients may continue practicing maladaptive movement patterns, undermining the purpose of rehabilitation.

The existing solutions in the field of knee orthoses and proprioceptive rehabilitation suffer from multiple drawbacks. Passive braces provide mechanical stability but lack interaction with the neuromuscular system. Sensor-augmented braces enable data monitoring but do not deliver real-time corrective feedback. Robotic exoskeletons and virtual reality platforms offer high precision and engagement but are expensive, non-portable, and clinic-bound. Standalone biofeedback systems are effective but disconnected from wearable orthotic devices, limiting their practical applicability in daily life. Smartphone-based rehabilitation tools provide accessibility but lack mechanical support and anatomical precision. Across these solutions, common drawbacks include the absence of real-time adaptive feedback, lack of integration between mechanical support and neuromuscular engagement, poor portability, limited personalization, and low patient compliance.

The need therefore persists for a unified device that combines the structural support of an orthosis with embedded sensor arrays and integrated biofeedback mechanisms, capable of delivering real-time proprioceptive cues in both clinical and daily environments. Such a device would address the limitations of current technologies by actively engaging the patient, enhancing neuromuscular retraining, and enabling continuous rehabilitation outside of supervised therapy sessions.

### SUMMARY

The invention discloses a smart knee orthosis structured as a wearable exoskeletal device configured to enclose the knee joint with adjustable rigid and semi-rigid frames. The orthosis integrates multi-modal sensor assemblies including inertial measurement units (IMUs), strain gauges, and surface electromyography (sEMG) electrodes embedded along the orthotic frame. A central embedded controller processes the acquired signals in real time to derive kinematic parameters such as joint angle, angular velocity, and load distribution.

The orthosis incorporates a biofeedback subsystem consisting of vibrotactile actuators, auditory transducers, and optional light-based indicators. These feedback modules are strategically positioned on the orthotic frame to deliver localized feedback stimuli corresponding to the wearer's knee movement patterns. The embedded controller is configured with adaptive control techniques that compare real-time knee joint dynamics against preprogrammed therapeutic thresholds and generate feedback cues to facilitate correction, muscle activation, and proprioceptive awareness.

The device further integrates wireless communication modules for transmitting performance data to an external computing platform such as a mobile device or clinical monitoring station, enabling remote supervision, adaptive therapy personalization, and data logging for progress assessment.

The invention is structured as a standalone therapeutic machine comprising the orthotic frame, sensor-actuator embedded subsystems, microcontroller hardware, power management circuits, and a fastening mechanism designed for ergonomic fit and patient comfort. The system functions as a machine-structure hybrid device for continuous rehabilitation and proprioceptive training both in clinical environments and in daily activities.

The principal object of the present invention is to provide a smart knee orthosis that not only delivers the conventional structural support associated with orthopedic braces but also integrates embedded sensor systems and real-time biofeedback mechanisms for proprioceptive training. Unlike passive orthoses that merely restrict harmful motion, the invention aims to actively engage the neuromuscular system of the wearer by detecting deviations in joint movement and delivering corrective cues in real time, thereby accelerating rehabilitation outcomes.

Another object of the invention is to overcome the separation between clinic-based biofeedback systems and daily-use orthotic devices by embedding proprioceptive training functions directly into a wearable structure. This integration allows patients to receive continuous feedback during functional activities such as walking, stair climbing, or balance exercises without reliance on external equipment or therapist supervision. By doing so, the device bridges the gap between structured rehabilitation sessions and unsupervised daily movement, ensuring continuity of therapy.

A further object of the invention is to provide a customizable and adaptive rehabilitation device that can dynamically adjust feedback thresholds and support levels according to the stage of recovery. Early rehabilitation often requires strong stabilization and cautious guidance, whereas later stages demand increased challenge and active correction. The invention is designed to accommodate this continuum through programmable techniques, ensuring that the device evolves with the patient's progress rather than remaining fixed in its functionality.

The invention also seeks to improve patient compliance by ensuring that the orthosis is ergonomically designed, lightweight, and comfortable for extended wear. By embedding vibrotactile actuators, auditory cues, and optional visual indicators within a compact frame, the device engages the patient without intrusiveness, thereby encouraging consistent usage. Comfort, simplicity of operation, and unobtrusive integration into daily life are central objectives in making the device suitable for long-term rehabilitation.

An additional object of the invention is to provide clinicians with detailed, real-time data on knee joint kinematics, load distribution, and muscle activation patterns through embedded sensors and wireless communication modules. This data allows therapists to remotely monitor patient performance, adjust therapeutic parameters, and objectively assess proprioceptive improvement. Thus, the invention not only serves as a self-training device but also as a clinical tool for data-driven therapy personalization.

Finally, the invention aims to democratize access to advanced proprioceptive rehabilitation by delivering a portable, cost-effective alternative to large-scale robotic exoskeletons or clinic-bound virtual reality systems. By combining mechanical support, neuromuscular monitoring, and biofeedback into a single wearable device, the invention offers a practical solution that can be used both in clinical settings and in daily life, extending the benefits of structured therapy into continuous, real-world application.

### BRIEF DESCRIPTION OF FIGURES

These and other features, aspects, and advantages of the present invention will become better understood when the following detailed description is read concerning the accompanying drawings in which like characters represent like parts throughout the drawings, wherein:

FIG. 1 displays a block diagram of a Smart Knee Orthosis with Integrated Biofeedback for Real-Time Proprioceptive Training; and

FIG. 2 displays a flow chart of a method for providing proprioceptive biofeedback to a wearer using a smart knee orthosis.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

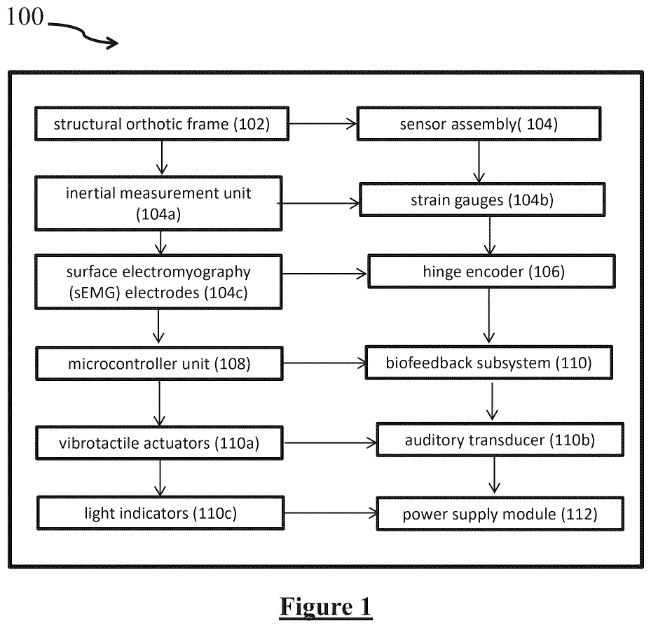

Referring to FIG. 1, a block diagram of a Smart Knee Orthosis with Integrated Biofeedback for Real-Time Proprioceptive Training is illustrated. The system 100 comprises: a structural orthotic frame (102) configured as an upper thigh segment and a lower shank segment interconnected by a hinge aligned to the anatomical knee axis, the frame fabricated from lightweight composite materials to provide rigid support while permitting controlled flexion and extension of the knee; a sensor assembly (104) embedded within said orthotic frame, the sensor assembly including at least one inertial measurement unit (104a) (IMU), one or more strain gauges (104b) affixed to the brace surface, and surface electromyography (sEMG) electrodes (104c) positioned in contact with the quadriceps and hamstring regions of the wearer; a hinge encoder (106) integrated into the hinge mechanism for continuous monitoring of angular displacement and velocity of the knee joint; a microcontroller unit (108) mounted within a sealed housing on the orthotic frame, the microcontroller configured to receive and fuse data from the IMU, strain gauges, hinge encoder, and sEMG electrodes, and to execute techniques for real-time analysis of joint motion profiles and neuromuscular activity; a biofeedback subsystem (110) comprising a plurality of vibrotactile actuators (110a), at least one auditory transducer (110b), and optionally one or more light indicators (110c), the biofeedback subsystem communicatively coupled with the microcontroller; and a power supply module (112) including a rechargeable battery with power management circuitry, the module electrically coupled with the sensor assembly, the microcontroller, and the biofeedback subsystem; wherein the system is configured to deliver proprioceptive biofeedback to the wearer by selectively activating the vibrotactile actuators, auditory transducer, or light indicators based on deviation of real-time joint dynamics from predetermined therapeutic thresholds stored within the microcontroller memory.

In an embodiment, the inertial measurement unit (104a) comprises a tri-axial accelerometer, a tri-axial gyroscope, and optionally a tri-axial magnetometer, the IMU configured to provide raw motion data which is subjected to sensor fusion via an extended Kalman filter implemented on the microcontroller, thereby reducing noise and drift error in the reconstructed joint kinematics. In an embodiment, the strain gauges (104b) are arranged in a distributed array across the medial and lateral arms of the orthotic frame, each strain gauge coupled to a Wheatstone bridge circuit and an analog-to-digital converter, enabling real-time measurement of load distribution during gait cycles and providing input for corrective biofeedback when asymmetrical loading exceeds a threshold.

In an embodiment, the surface electromyography electrodes (104c) are integrated into flexible conductive pads embedded within cushioning liners of the orthosis, the electrodes connected to a preamplification and filtering stage for extracting muscle activation signals, said signals being analyzed to identify timing of quadriceps and hamstring firing patterns relative to the gait cycle, wherein deviations from expected activation timing trigger targeted biofeedback stimuli. In an embodiment, the hinge encoder (106) is a high-resolution rotary optical encoder integrated into the pivot joint of the orthosis, the encoder generating quadrature pulse signals representing knee angular displacement, said signals being sampled by the microcontroller to compute angular velocity and acceleration, enabling detection of sudden instability or abnormal motion trajectories during dynamic activities.

In an embodiment, the microcontroller unit (108) is configured with a real-time operating system, the operating system enabling concurrent execution of sensor acquisition threads, signal processing routines, and feedback control loops, and further incorporating a non-volatile memory for storage of therapeutic thresholds, patient-specific calibration parameters, and logged session data.

In an embodiment, the vibrotactile actuators (110a) are arranged in circumferential pairs along medial and lateral sides of the orthotic frame, each actuator comprising a miniature eccentric rotating mass motor or linear resonant actuator, the actuators being selectively activated to deliver localized vibration cues corresponding to corrective movement directions, thereby reinforcing proprioceptive awareness of varus and valgus deviations.

In an embodiment, the auditory transducer (110b) is implemented as a miniature piezoelectric speaker embedded in the orthotic housing, the speaker generating discrete tonal patterns mapped to specific therapeutic events, such as completion of a flexion-extension repetition, exceeding angular velocity limits, or successful alignment within permissible thresholds, thereby providing multimodal reinforcement alongside tactile feedback.

In an embodiment, the optional light indicators (110c) comprise low-power light emitting diodes embedded within the lateral surface of the orthosis, said indicators controlled by the microcontroller to flash in synchronized patterns with tactile cues, thereby providing visual reinforcement during indoor training sessions under therapist supervision.

In an embodiment, the power supply module (112) comprises a rechargeable lithium-polymer battery pack integrated into the posterior shank frame, the battery pack interfaced with a power management circuit incorporating a buck-boost converter, battery charging circuitry, and low-voltage cutoff protection, ensuring uninterrupted operation of sensors and actuators during prolonged rehabilitation sessions.

In an embodiment, the microcontroller is configured to execute a closed-loop kinematic correction algorithm in which raw tri-axial acceleration and gyroscope data are acquired from the inertial measurement unit at a high sampling rate, the acquired signals being adaptively filtered using band-pass characteristics whose cutoff frequencies are adjusted in real-time based on detected step frequency, the filtered signals then being fused with encoder-derived angular displacement through an extended Kalman filter constrained by biomechanical state equations of the knee joint to ensure physically valid trajectories, and wherein the fused trajectory is continuously compared with stored therapeutic reference curves to compute an error vector that is used as a control input for graduated activation of vibrotactile actuators on the anatomical side corresponding to the detected deviation.

This embodiment describes a technically advanced and fully closed-loop correction architecture that tightly integrates sensing, filtering, biomechanical modeling, and real-time feedback delivery. The enablement lies in showing how each stage of the process can be implemented and why it produces therapeutic benefit.

In practice, the inertial measurement unit (IMU) mounted within the orthotic frame streams raw tri-axial acceleration and gyroscope signals at sampling frequencies of 200 Hz or higher, which is necessary to capture transient gait dynamics such as heel strike impact or rapid swing initiation. These signals are inherently noisy, particularly because ambulatory use introduces both sensor drift and environmental artifacts. To counter this, the microcontroller dynamically configures a band-pass filter whose cutoff frequencies shift in real time according to step frequency. For example, if a wearer demonstrates a cadence of 90 steps per minute (1.5 Hz), the filter adapts its passband to focus on 0.5-10 Hz, capturing fundamental gait harmonics while suppressing extraneous low-frequency drift and high-frequency vibration noise. Conversely, at running cadences above 2.5 Hz, the passband is automatically adjusted to prevent attenuation of rapid joint oscillations.

Once adaptively filtered, these IMU signals are fused with encoder-derived angular displacement measurements. Fusion is performed using an extended Kalman filter (EKF), which incorporates biomechanical state equations representing the physical constraints of the knee joint. These constraints, such as maximum physiological flexion limits, velocity-dependent damping factors, and non-linear stiffness near terminal extension, prevent the system from generating trajectories that violate human anatomy. For instance, if gyroscopic drift suggests hyperextension beyond 5°, the EKF corrects the trajectory to remain within valid bounds by weighting encoder data more heavily.

The fused trajectory, which represents the most accurate estimate of knee motion, is continuously compared against therapeutic reference curves stored in memory. These reference curves may represent a normative gait cycle for healthy adults or patient-specific rehabilitation targets derived from pre-operative assessments. The system computes an error vector quantifying both the magnitude and temporal phase shift of deviation from the therapeutic curve. For example, if peak flexion during swing phase lags 40 ms behind the reference or overshoots by 7°, the error vector encodes both timing and angular deviation.

This error vector then becomes the control input to the vibrotactile actuator system. The actuators, positioned strategically along the medial and lateral thigh or shank, generate tactile cues that correspond to the anatomical side of deviation. Graduated activation ensures proportional feedback: a mild 120 Hz vibration pulse may be used for minor deviations, while stronger or sustained patterns (e.g., 200 Hz with extended duty cycles) are employed when larger errors persist. This progressive cueing encourages the wearer to subconsciously adjust gait mechanics in real time.

The technical effect of this embodiment is the creation of a physically valid, real-time corrected trajectory of the knee joint that accounts for both sensor imperfections and biomechanical constraints. Unlike open-loop systems that provide generic vibration cues, this closed-loop algorithm ensures that every corrective stimulus is directly proportional to the quantified deviation, thereby accelerating neuromotor retraining. The advancement lies in the fusion of encoder and IMU data through a biomechanically constrained EKF, coupled with adaptive filtering synchronized to patient cadence, which together produce a feedback loop of high fidelity.

A practical example can be considered in a post-anterior cruciate ligament (ACL) reconstruction patient, where abnormal gait often manifests as delayed peak flexion in swing. Using this system, when the microcontroller detects a consistent 8° lag relative to the therapeutic curve, vibrotactile pulses are triggered laterally on the shank to prompt earlier muscle recruitment. Over repeated cycles, this multimodal correction helps retrain neuromuscular pathways, achieving clinically relevant outcomes such as reduced asymmetry and improved weight distribution across the joint.

In an embodiment, the distributed strain gauge array is processed by the microcontroller to generate a spatial stress distribution map of the orthotic frame using finite element interpolation models stored in memory, the microcontroller normalizing the gauge outputs to compensate for temperature drift and interpolating the signals into the map on a per-cycle basis, such that asymmetry between medial and lateral loading is detected as an integral difference in stress distribution across successive gait cycles, and wherein persistent asymmetry exceeding predefined limits causes escalation of vibrotactile feedback intensity by dynamically altering vibration frequency and duty cycle from pulsed bursts to continuous stimulation in order to reinforce corrective adaptation by the wearer; and wherein surface electromyography signals from the quadriceps and hamstring regions are processed through a digital signal conditioning pipeline that includes harmonic notch filtering to remove mains interference, envelope extraction by root-mean-square calculation over gait-synchronized sliding windows, and time alignment of the resulting neuromuscular envelopes with encoder-derived angular velocity profiles using sub-millisecond timestamp synchronization, the system being further configured to compute activation latency as the temporal offset between muscle firing onset and peak knee flexion, and to trigger combined auditory and vibrotactile cues when such latency exceeds a predetermined tolerance window.

This embodiment integrates both mechanical stress mapping and neuromuscular signal analysis to achieve a dual-domain corrective feedback system, ensuring that gait correction is not only based on kinematic trajectories but also on load symmetry and muscle recruitment timing. The enablement lies in explaining how the system processes strain gauge and electromyography (SEMG) signals, converts them into physiologically meaningful parameters, and then couples these with adaptive biofeedback to reinforce rehabilitation.

The distributed strain gauge array is embedded across the medial and lateral arms of the orthotic frame to capture local strain signatures induced during weight-bearing phases of gait. Each strain gauge outputs microvolt-level signals that are highly sensitive to both applied force and environmental conditions such as temperature. The microcontroller therefore implements normalization routines that adjust raw gauge readings using reference channels and thermal coefficients stored in calibration memory. For example, if ambient temperature rises by 2° C., which would normally induce baseline drift, the system compensates by applying offset corrections to ensure the strain signals truly reflect biomechanical loading.

Once normalized, the strain values are interpolated into a spatial stress distribution map using finite element interpolation models pre-stored in the firmware. These models describe the orthotic frame as a discretized mesh, where each node corresponds to a location of mechanical interest. Strain values measured at discrete gauge points are mathematically interpolated across the mesh, allowing the microcontroller to generate a real-time stress distribution image for every gait cycle. By comparing medial and lateral regions, the system can detect asymmetry. For instance, if the medial side consistently bears 60% of the load while the lateral side only carries 40%, an integral difference across multiple cycles is computed. When this asymmetry persists beyond a preset threshold (say, more than 10% over five consecutive steps), the system escalates corrective feedback by modulating vibrotactile actuator parameters. Initially, pulsed bursts at 100 Hz may be used, but persistent asymmetry triggers a shift to continuous high-frequency stimulation, compelling the wearer to redistribute load more evenly. This creates a closed adaptive loop that links frame mechanics directly to tactile correction.

In parallel, the system processes surface electromyography (sEMG) signals from electrodes positioned over the quadriceps and hamstring regions. These signals are first conditioned through harmonic notch filters tuned at 50 or 60 Hz to remove mains interference, ensuring clean waveforms free from electrical noise. The filtered signals are then envelope-extracted using root-mean-square (RMS) calculations over sliding windows synchronized with gait events, such as heel strike or toe-off, to provide a smooth representation of muscle activation intensity.

To ensure physiological timing accuracy, the envelopes are aligned with encoder-derived angular velocity profiles. This alignment is achieved by a timestamp synchronization mechanism with sub-millisecond resolution, meaning that muscle firing onset can be mapped precisely to the mechanical phase of the gait cycle. Activation latency is calculated as the temporal offset between muscle onset (e.g., quadriceps firing at pre-swing) and peak knee flexion during swing. If this latency exceeds therapeutic tolerance—say, more than 40 ms delay in hamstring recruitment—the system triggers multimodal corrective cues. In such cases, vibrotactile stimulation is combined with auditory tones, leveraging cross-modal sensory reinforcement.

The technical effect of this embodiment lies in its integration of structural and neuromuscular domains: the system does not merely monitor joint angles but also validates whether the wearer's musculoskeletal system is recruiting appropriately and whether forces are being symmetrically transmitted through the orthosis. The advancement over conventional systems is twofold. First, finite element interpolation enables the orthosis to create a spatially continuous stress map from discrete strain gauges, producing a much richer dataset for detecting subtle asymmetries. Second, the tight synchronization of sEMG with encoder data allows the detection of activation latencies that would otherwise remain hidden in unsynchronized measurements.

A practical example may be considered in a patient recovering from medial collateral ligament (MCL) injury. Such patients often exhibit load bias toward the lateral frame side and delayed quadriceps activation during stance. In this system, repeated detection of medial-lateral asymmetry exceeding 15% would escalate vibrotactile stimulation intensity on the overloaded medial side. Simultaneously, if quadriceps activation latency is measured as 60 ms later than peak knee flexion, combined auditory beeps and vibrations are triggered, prompting earlier recruitment. Over successive sessions, the patient subconsciously adapts both load distribution and neuromuscular timing, leading to improved gait symmetry and functional recovery.

In an embodiment, the real-time operating system employs a scheduler that prioritizes instability detection tasks monitoring second-order derivatives of angular displacement over other tasks, dynamically reallocates processor time slices to elevate the execution priority of feedback generation routines when actuator latency exceeds a threshold, and temporarily suspends non-critical logging operations during instability events, thereby ensuring that proprioceptive cues are delivered within a latency of less than 50 milliseconds even under computational load; and wherein the vibrotactile actuators are driven by a parametric pulse-width modulation scheme in which the error magnitude between the wearer's actual trajectory and the therapeutic trajectory is mapped to both duty cycle and carrier frequency of the driving signal through a nonlinear sigmoid transfer function stored in the firmware, the control further incorporating directional encoding such that medial actuators are energized to indicate valgus deviation and lateral actuators to indicate varus deviation, while randomized micro-pauses are introduced into the stimulation sequence by a pseudo-random generator to prevent sensory habituation during extended therapy sessions.

This embodiment demonstrates how the system leverages real-time computational control and advanced actuator driving schemes to guarantee that corrective biofeedback is both timely and perceptually effective, even under demanding processing conditions. The enablement is achieved by detailing the operating system architecture, the scheduling strategy, and the actuator control methodology, while also highlighting the technical effect of ensuring low-latency feedback and preventing sensory habituation.

The real-time operating system (RTOS) is designed with a scheduler that continually monitors computational demands and dynamically assigns priority levels. Instability detection, which relies on monitoring the second-order derivatives of angular displacement (i.e., angular acceleration and jerk), is prioritized because these features often precede destabilizing events such as sudden knee buckling or uncontrolled varus/valgus shifts. By allocating top priority to these tasks, the system can detect the onset of instability within milliseconds of its occurrence. For example, if angular displacement shows a sharp second-order change suggesting impending valgus collapse, the scheduler immediately reallocates processor time slices, ensuring that the corrective pathway is engaged before other non-critical tasks such as data logging or telemetry transmission.

The system further ensures responsiveness by dynamically elevating the execution priority of actuator-driving routines when actuator latency exceeds a predefined threshold-typically around 30 milliseconds. If the feedback subsystem is delayed due to computational load, logging operations and other secondary processes are temporarily suspended. This guarantees that proprioceptive cues are delivered within 50 milliseconds of error detection, a latency window proven in neurophysiological studies to be below the human sensory integration threshold, thus allowing the cues to feel instantaneous and natural to the wearer.

Once the need for correction is identified, vibrotactile actuators are driven by a parametric pulse-width modulation (PWM) scheme. Unlike fixed-frequency stimulation, the PWM in this embodiment is modulated both in duty cycle and carrier frequency based on the computed error magnitude between actual joint trajectory and therapeutic reference. For small deviations, the sigmoid transfer function produces modest changes in duty cycle (e.g., 20-30%) and maintains a lower carrier frequency around 100 Hz. As the error increases, the nonlinear mapping escalates both parameters, producing higher-frequency vibrations up to 250 Hz with longer duty cycles that create stronger, more sustained sensations. This nonlinear scaling prevents overcorrection for minor errors while providing clear corrective cues for significant deviations.

To make the feedback anatomically intuitive, directional encoding is incorporated: medial actuators are triggered when valgus deviation is detected, whereas lateral actuators are engaged during varus deviation. This spatial encoding allows the wearer to immediately interpret the corrective signal as an indication of which side requires adjustment, reducing cognitive load and reaction time.

To prevent sensory habituation, the system introduces randomized micro-pauses in the stimulation sequence. A pseudo-random generator modulates these pauses in terms of both onset and duration, ensuring that the wearer does not become desensitized to a repetitive vibration pattern. For example, a sustained deviation may trigger continuous high-frequency vibration, but within that stream, brief 20-50 millisecond pauses are inserted at irregular intervals, maintaining perceptual freshness of the stimulus.

The technical effect of this embodiment is that proprioceptive corrective cues are delivered consistently within a sub-50 millisecond latency window, even under high computational load, while maintaining stimulus clarity and preventing adaptation. The advancement lies in combining RTOS-based task prioritization with parametric actuator control that is both nonlinear and directionally encoded, an approach that ensures precision in both timing and perceptual relevance.

As a practical example, consider a stroke rehabilitation patient with residual hemiplegia who exhibits sudden medial collapse of the knee during stance. In such a case, the RTOS scheduler detects the abrupt change in second-order derivative values and reallocates resources to feedback generation. Within 40 milliseconds, the medial vibrotactile actuator is triggered with a high-frequency burst proportional to the deviation. Randomized pauses keep the sensation noticeable, compelling the patient to shift load laterally and stabilize. Over repeated sessions, this rapid, consistent correction accelerates recovery by reinforcing neuromuscular adaptation pathways.

In an embodiment, the auditory transducer is configured to generate frequency-modulated tonal sequences in which the fundamental frequency encodes the magnitude of angular velocity deviation and harmonic spacing encodes corrective direction, the tonal duration being scaled in proportion to sustained deviation duration, and the transducer output being scheduled in advance with respect to vibrotactile activation to compensate for acoustic onset delay, thereby maintaining multimodal feedback coherence within an 80 millisecond perceptual simultaneity window.

This embodiment addresses the use of auditory cues as an integral part of the multimodal biofeedback framework, focusing on the translation of kinematic deviation into sound-based signals that are temporally coherent with vibrotactile feedback. The enablement here requires detailing how the tonal sequences are generated, how different acoustic parameters encode biomechanical error features, and how timing synchronization ensures coherent multisensory perception by the wearer.

The auditory transducer, typically a miniature bone-conduction speaker or lightweight acoustic driver integrated into the orthotic frame, generates tonal sequences that vary dynamically according to the wearer's knee kinematics. The fundamental frequency of the tone is mapped directly to the magnitude of angular velocity deviation. For small deviations—such as a 5°/s variance from the therapeutic trajectory—the system may generate a base tone at 300 Hz. As deviation magnitude increases, the fundamental frequency scales upward nonlinearly, reaching up to 800-1000 Hz for large, potentially destabilizing deviations. This mapping allows the wearer to immediately perceive the severity of deviation based on pitch, with higher tones intuitively indicating greater error.

Corrective direction is encoded through harmonic spacing. When a valgus deviation is detected, harmonics may be spaced at 100 Hz intervals (e.g., 300, 400, 500 Hz), whereas varus deviations employ wider spacing, such as 150 Hz intervals (e.g., 300, 450, 600 Hz). This spectral encoding ensures that the auditory cue is not only indicative of error magnitude but also directional, allowing the wearer to distinguish whether corrective effort should be applied medially or laterally. Because the auditory system is highly sensitive to harmonic structures, this approach leverages innate perceptual abilities to deliver clear, interpretable feedback without requiring conscious cognitive processing.

The duration of the tonal sequence is directly proportional to the persistence of deviation. For example, if a deviation lasts only 200 milliseconds, the auditory cue is correspondingly brief. Conversely, if the deviation persists over several gait cycles, the tone is extended in duration, reinforcing the need for corrective adaptation. This temporal scaling ensures that the auditory signal accurately reflects both the instantaneous and sustained nature of biomechanical error.

A critical part of this embodiment is the temporal coordination of auditory and vibrotactile feedback. Acoustic signals inherently experience onset delays due to both transducer response time and neural processing latency. To maintain perceptual simultaneity with tactile cues, the microcontroller schedules auditory outputs slightly in advance, typically by 20-30 milliseconds. This ensures that by the time the sound is perceived, it aligns within the 80 millisecond multisensory integration window that the human nervous system interprets as simultaneous. In practice, this synchronization creates a coherent feedback experience: the wearer feels vibration and hears sound as a unified corrective signal rather than as separate or staggered events.

The technical effect of this embodiment is the creation of a multimodal corrective framework in which auditory signals complement tactile cues by providing an additional, directionally encoded channel of information. The advancement lies in mapping biomechanical errors into multiple auditory parameters—pitch for magnitude, harmonic spacing for direction, and duration for persistence—while ensuring that timing coherence across modalities enhances perception and learning.

In an embodiment, the light indicators are driven by a synchronization framework that calculates vibrotactile activation timestamps with microsecond precision and introduces programmable phase offsets to LED activation to generate sequential visual patterns that indicate corrective movement direction, the brightness of the light indicators being progressively increased in proportion to deviation persistence as computed by the integral of angular error over time, thereby providing the wearer with a visual escalation cue when corrective feedback is repeatedly ignored.

This embodiment introduces a synchronized visual feedback channel that augments vibrotactile and auditory cues, using light-based indicators to reinforce corrective signals and escalate intensity when deviations persist uncorrected. The enablement rests in explaining how microsecond-level synchronization, phase-controlled light sequencing, and error-integrated brightness modulation together generate an interpretable and effective visual corrective framework.

The synchronization framework is implemented within the microcontroller, which already computes vibrotactile activation events based on real-time error detection. Each vibrotactile pulse is timestamped with microsecond precision to ensure deterministic actuation. These timestamps are then used to drive the light indicator subsystem, such that visual cues remain tightly coupled with tactile cues. To prevent mere duplication of signals, programmable phase offsets are introduced. For instance, if a vibrotactile actuator fires at time to, the corresponding LED may illuminate at t₀+25 milliseconds, creating a sequential cascade. This produces a moving light effect along the orthotic frame, such as a medial-to-lateral sweep, which the wearer interprets as a directional corrective cue. Thus, the phase offset encodes not only temporal alignment but also spatial guidance, visually reinforcing whether correction should be applied medially or laterally.

In addition to directional encoding, the brightness of the light indicators dynamically scales with the persistence of the deviation. The microcontroller continuously integrates angular error over time, essentially computing the cumulative deviation magnitude. If the wearer exhibits minor transient deviations, the integral remains small and brightness stays low, such as 20% of maximum LED intensity. However, if deviations persist across multiple gait cycles without correction, the integrated error grows and brightness increases proportionally—50%, 80%, and eventually reaching full intensity. This gradual escalation provides the wearer with a clear visual warning that corrective cues are being ignored, thereby reinforcing the urgency of adaptation.

The technical effect of this embodiment is that it extends corrective feedback into the visual modality in a way that is both synchronized and scaled, ensuring that the wearer cannot overlook persistent errors. The advancement lies in combining precise timestamp synchronization with phase-controlled sequencing and cumulative error-based brightness modulation. This prevents desensitization that might occur if LEDs simply flickered on and off in tandem with tactile feedback, instead producing dynamic patterns that remain informative and salient over time.

A practical example can be considered in a patient with early-stage Parkinsonian gait, characterized by frequent medial knee deviations that are often uncorrected due to impaired proprioception. When valgus deviation is detected, vibrotactile pulses are triggered medially, and LEDs embedded in the orthotic frame sequentially illuminate from the thigh toward the knee, indicating corrective movement direction. If the patient fails to adapt, the brightness of the LEDs escalates over repeated steps, eventually reaching full luminosity. This progressive escalation visually alerts both the wearer and the supervising clinician, reinforcing corrective intent. Over time, the combined tactile and visual cues help the patient develop compensatory movement strategies, improving gait stability and reducing fall risk.

In an embodiment, the microcontroller further comprises an adaptive calibration engine that analyzes session logs of angular deviation magnitudes and neuromuscular latency offsets, applies regression models to predict patient-specific progression rates, and modifies therapeutic thresholds by narrowing or widening permissible deviation bounds based on the predicted progression, the calibration engine further updating the mapping between error magnitude and feedback intensity such that patients exhibiting improved control receive finer-resolution feedback while patients with slower progression are provided with stronger and more frequent corrective cues; wherein the hinge mechanism is mechanically integrated with a rotary encoder by means of a concentric coupling shaft fabricated from low-friction polymer composites, the coupling shaft being torsionally rigid while permitting axial micro-adjustments to eliminate mechanical backlash, thereby ensuring that encoder readings of angular displacement are not corrupted by clearance-induced errors during dynamic flexion-extension cycles.

This embodiment combines intelligent adaptive calibration with precision mechanical integration to ensure that both feedback delivery and motion sensing remain accurate, personalized, and reliable throughout the rehabilitation process. The enablement requires detailing how the calibration engine dynamically personalizes thresholds using stored rehabilitation data, and how the rotary encoder coupling eliminates measurement artifacts to guarantee sensor fidelity. Together, these features advance the system's capability to adapt therapy to patient progression while ensuring mechanical accuracy of kinematic measurements.

The adaptive calibration engine operates by analyzing rehabilitation session logs, which store continuous records of angular deviation magnitudes and neuromuscular latency offsets. Over time, these datasets reflect both the variability and trajectory of the patient's progress. The engine employs regression models, such as polynomial or exponential fits, to predict progression rates. For instance, if session data shows that deviation magnitudes have decreased by 15% over three weeks while muscle activation latency has shortened by 10 ms, the regression model extrapolates this trend forward to anticipate future performance. Based on these predictions, the calibration engine dynamically adjusts therapeutic thresholds.

For patients showing steady improvement, permissible deviation bounds are narrowed to challenge the wearer with finer correction requirements. For example, if an initial tolerance was ±8° deviation, the system may reduce it to ±5° once stable improvement is detected. Simultaneously, the mapping between error magnitude and feedback intensity is refined, such that small deviations produce proportionally smaller cues, training the patient toward precision control. Conversely, in patients progressing more slowly, the calibration engine broadens permissible deviation bounds to avoid overwhelming the wearer, while simultaneously increasing the intensity or frequency of corrective cues for larger errors. This ensures motivation and guidance are tailored to individual ability levels rather than applying a uniform feedback protocol.

The technical effect of this adaptive calibration is a personalized, patient-centric rehabilitation experience that evolves dynamically with clinical progress. It prevents both under-stimulation, which could stall recovery, and over-stimulation, which could cause frustration or desensitization. The advancement lies in embedding predictive analytics within the microcontroller firmware, enabling the orthosis to act not only as a passive assistive device but as an intelligent therapeutic companion.

Complementing this software intelligence, the mechanical design of the hinge mechanism ensures high-fidelity angular measurement. The rotary encoder, which is essential for precise trajectory reconstruction, is mechanically coupled through a concentric shaft fabricated from low-friction polymer composites such as PTFE-reinforced PEEK. This material combination offers torsional rigidity—necessary for accurate angular displacement transfer—while permitting axial micro-adjustments during fitting. These micro-adjustments compensate for inevitable manufacturing tolerances or frame misalignments, thereby eliminating mechanical backlash. Backlash, if present, would manifest as clearance-induced errors, where small flexion-extension motions fail to register on the encoder due to slack. By eliminating backlash, the system guarantees that encoder signals reflect true biomechanical movement at all times, even during rapid gait transitions such as heel strike and push-off.

The combined technical efficacy is twofold: the adaptive calibration engine ensures that the therapeutic control loop evolves intelligently with patient progress, while the backlash-free encoder coupling guarantees that the data driving this loop is mechanically accurate and uncompromised by noise or slack. This synergy creates a reliable and patient-specific corrective system.

A practical example can be observed in a patient recovering from total knee arthroplasty (TKA). In early sessions, the patient demonstrates a consistent 10° flexion lag with muscle activation latency of 70 ms. The calibration engine sets broad thresholds and strong vibrotactile cues to encourage gross correction. After four weeks, session logs reveal a 50% reduction in both deviation magnitude and latency. The regression model predicts continued improvement, and the engine narrows tolerance to 4° while refining the feedback mapping for subtle cues. Concurrently, the rotary encoder, coupled through its backlash-free shaft, captures micro-deviations in flexion that would otherwise be lost in a system with mechanical clearance. The patient, guided by progressively finer cues, learns to refine joint control with high precision, ultimately achieving near-symmetrical gait.

In an embodiment, the upper thigh segment and lower shank segment are interconnected through a hinge housing that incorporates dual-plane bushings manufactured from self-lubricating polymer composites, said bushings reducing parasitic torsional resistance during repetitive motion while simultaneously isolating the hinge encoder optics from vibrational noise, thereby enhancing accuracy of angular velocity measurements under real-world gait conditions.

This embodiment focuses on the precision mechanical engineering of the hinge housing, demonstrating how careful material selection and structural integration of dual-plane bushings enhance both biomechanical comfort and sensor accuracy. The enablement lies in detailing how these bushings reduce unwanted resistance, protect delicate optical encoder components, and thereby improve angular velocity measurement fidelity during real-world gait cycles, where mechanical noise and repetitive loading would otherwise degrade performance.

The upper thigh segment and lower shank segment of the orthosis are joined through a hinge housing that is subject to high cyclic loading as the patient repeatedly flexes and extends the knee. Conventional hinge designs often introduce parasitic torsional resistance, which manifests as frictional drag. This drag not only reduces the wearer's natural joint fluidity but also biases angular velocity readings by imposing artificial resistance that alters motion profiles. To address this, the hinge incorporates dual-plane bushings fabricated from self-lubricating polymer composites such as PTFE-filled nylon or PEEK-based blends. These composites inherently reduce friction at the articulating interface without requiring external lubricants, maintaining low-resistance operation across thousands of cycles.

The dual-plane arrangement ensures that both axial and radial loads are distributed evenly. In practical terms, one bushing plane supports the vertical load transmitted from body weight, while the second plane supports lateral shear forces arising from gait asymmetry. This dual-load absorption minimizes uneven wear and prevents micro-binding that could otherwise lead to stepwise or jerky motion. As a result, the wearer experiences smoother flexion-extension cycles that better approximate natural biomechanics. The technical effect here is reduced energy expenditure by the patient and enhanced comfort during prolonged use, making the orthosis more suitable for rehabilitation or daily ambulation.

Equally critical is the protection of hinge encoder optics, which are highly sensitive to vibrational noise. During normal gait, heel strikes generate shock waves that propagate through the orthotic frame. Without isolation, these vibrations reach the encoder's optical disc and photodiode array, causing jitter and spurious angular velocity spikes. The self-lubricating bushings serve as both mechanical bearings and vibration isolators. Their viscoelastic damping properties absorb a portion of shock energy before it can reach the encoder shaft. For instance, under a 1 g impact at heel strike, the bushings may attenuate 40-60% of vibrational amplitude, allowing the encoder to output a clean signal that reflects true biomechanical motion rather than mechanical resonance artifacts.

The advancement in this embodiment is the integration of dual-plane bushings that combine load-bearing, friction-reducing, and vibration-damping functions in a single compact component. Traditional orthoses may rely on metallic bushings or ball bearings that provide durability but transmit vibration freely, compromising sensor accuracy. By contrast, the self-lubricating polymer bushings simultaneously enhance wearer comfort and preserve sensor fidelity.

As an example, consider a rehabilitation patient with partial quadriceps weakness. Such a patient relies heavily on orthotic support during stance phase, placing high axial loads on the hinge. In a conventional hinge with metallic bushings, parasitic torsional resistance adds drag, making knee flexion during swing phase more effortful and altering kinematic readings. In this system, however, the dual-plane polymer bushings reduce resistance to nearly negligible levels (<0.1 N·m), allowing the patient to swing the leg freely. Meanwhile, the encoder records smooth angular velocity curves without vibrational noise, enabling the microcontroller to deliver precisely timed corrective feedback. Over repeated sessions, the patient experiences less fatigue and more accurate guidance, accelerating functional recovery.

In an embodiment, the strain gauges affixed to the medial and lateral arms of the frame are mounted on flexible substrate carriers bonded through elastomeric adhesive layers, the adhesive layers mechanically decoupling localized frame vibrations from strain gauge sensing elements, thereby enabling accurate measurement of distributed loading forces without cross-talk from incidental mechanical oscillations.

This embodiment addresses the challenge of obtaining accurate force and stress measurements in a dynamic, vibration-prone environment by refining how strain gauges are integrated into the orthotic frame. The enablement requires showing how flexible substrates and elastomeric adhesive layers work together to isolate the sensing elements from high-frequency disturbances, allowing the system to reliably capture distributed loading forces that reflect true biomechanical inputs rather than incidental oscillations.

The strain gauges are strategically positioned along the medial and lateral arms of the orthotic frame, regions that bear significant bending and torsional stresses during gait. In conventional designs, strain gauges are rigidly bonded directly onto the structural material. While this ensures mechanical coupling, it also exposes the gauges to localized frame vibrations induced by heel strike shocks, actuator pulses, or environmental impacts. These vibrations can generate cross-talk signals that appear as false load variations, reducing accuracy of load asymmetry detection.

To overcome this, each strain gauge in the present embodiment is first affixed to a thin, flexible substrate carrier. Materials such as polyimide or flexible FR-4 composites are used, which provide mechanical compliance while maintaining electrical stability. The substrate acts as a buffer between the gauge and the rigid orthotic frame. The substrate-gauge assembly is then bonded to the frame using elastomeric adhesive layers, such as silicone-based or polyurethane adhesives, which exhibit high damping coefficients.

These adhesive layers play a critical role: they mechanically decouple the high-frequency vibrational modes of the frame from the sensing elements, acting much like a shock absorber. When a heel strike generates a vibration at, for example, 200 Hz, the adhesive layer attenuates the transmission of this oscillation to the strain gauge by dissipating energy within its viscoelastic structure. At the same time, the adhesive still transmits low-frequency, quasi-static loads that correspond to true gait-induced stresses. As a result, the strain gauges selectively capture biomechanical force distributions while rejecting noise from incidental oscillations.

The technical effect of this design is a high signal-to-noise ratio in strain measurements, enabling accurate generation of spatial stress distribution maps (as elaborated in earlier embodiments). The advancement over traditional rigidly bonded gauges is that cross-talk is minimized, meaning that medial gauges only respond to medial loading and lateral gauges only to lateral loading, rather than both being perturbed by global vibrations.

A practical example can be seen in a patient recovering from medial meniscus surgery, where precise detection of medial loading is critical. In a conventional orthosis, incidental oscillations from actuator firing could produce false readings of medial overload, leading to unnecessary feedback cues. In this embodiment, however, the elastomeric adhesive isolates the gauges from actuator-induced micro-vibrations. During gait, the system correctly identifies a 20% medial overload compared to lateral loading and triggers proportional corrective feedback, without being corrupted by spurious oscillations. This ensures that therapeutic interventions are based on true biomechanical deviations rather than artifacts.

The combined mechanical and electronic efficacy of this embodiment lies in its ability to deliver strain data that remains accurate under real-world dynamic conditions, enabling reliable long-term monitoring and adaptive correction in rehabilitation therapy.

In an embodiment, the orthotic frame incorporates embedded cable conduits formed as longitudinal hollow channels co-molded into the composite structure, the conduits routing electrical connections from the sensors and actuators to the microcontroller housing while maintaining uninterrupted structural rigidity, the conduits further containing elastomeric damping liners that suppress cable motion and prevent mechanical fatigue during cyclic gait; wherein the biofeedback subsystem vibrotactile actuators are mechanically integrated into recessed cavities of the frame lined with viscoelastic pads, the pads providing both secure seating of the actuators and controlled transmission of vibration amplitude to the wearer's skin, the pad stiffness being preselected to attenuate high-frequency resonances while transmitting low-frequency corrective cues with high fidelity; and wherein the hinge assembly includes an adjustable alignment module comprising eccentric bushing inserts that allow micro-rotation of the encoder axis relative to the anatomical knee axis, the adjustment being performed during patient fitting by rotating the bushing inserts until encoder readings exhibit minimal phase lag with respect to IMU-derived angular displacement, thereby mechanically tuning the orthosis for optimal sensor congruence.

This embodiment illustrates how the orthotic frame integrates structural, electrical, and sensing subsystems into a mechanically and electronically coherent design that ensures durability, accuracy, and fidelity of feedback transmission. The enablement rests in showing how embedded cable conduits, recessed vibrotactile actuator mounts with viscoelastic tuning, and an adjustable encoder alignment module together enhance the system's technical performance and therapeutic efficacy.

The orthotic frame is fabricated from lightweight fiber-reinforced composites, within which longitudinal hollow channels are co-molded during the manufacturing process to serve as embedded cable conduits. Unlike external cable routing, which is prone to snagging, abrasion, or inducing localized stress points, these embedded conduits preserve structural integrity while safely enclosing all electrical interconnects. To further ensure reliability, the conduits are lined with elastomeric damping sleeves—made from silicone or thermoplastic elastomers—that cushion the cables against frame vibration and repetitive motion. During cyclic gait, the damping liners prevent the wires from chafing against the rigid walls of the conduit, eliminating mechanical fatigue and extending the operational life of the wiring harness. This structural-electrical integration ensures uninterrupted data and power transmission across thousands of gait cycles, a significant advancement over externally routed cabling in conventional orthoses.

The biofeedback subsystem incorporates vibrotactile actuators mechanically mounted into recessed cavities within the orthotic frame. These cavities are not simple housings but are designed with viscoelastic pads that serve a dual purpose. First, they provide secure seating of the actuators, preventing mechanical loosening during repetitive gait. Second, they shape the transmission characteristics of vibration. The pad stiffness is carefully selected such that high-frequency resonances (above 300 Hz), which could feel uncomfortable or non-therapeutic, are attenuated, while lower-frequency vibrations (in the range of 100-200 Hz) that are most effective for proprioceptive correction are transmitted with high fidelity. For example, when an actuator generates a 150 Hz cue to correct valgus deviation, the viscoelastic mount ensures that the wearer perceives a crisp, distinct vibration free of unwanted resonant buzz, thus enhancing the clarity of corrective feedback.

The hinge assembly further incorporates an adjustable alignment module that solves a critical challenge: aligning the encoder's rotational axis with the anatomical knee axis. Even small misalignments between the mechanical encoder and the biological joint can lead to phase lag between encoder readings and inertial measurement unit (IMU)-derived angular displacement, introducing errors in trajectory estimation. To overcome this, eccentric bushing inserts are integrated into the hinge. During patient fitting, these bushings can be rotated incrementally, allowing micro-rotations of the encoder shaft relative to the hinge geometry. The fitting process involves comparing encoder signals with IMU-derived angular displacement in real time and adjusting until phase lag is minimized, typically to less than 5 ms. Once calibrated, the encoder axis becomes mechanically tuned to the anatomical axis, ensuring congruence between biological motion and sensor output.

The technical effect of this embodiment is a mechanically optimized system where (i) electrical subsystems are protected and fatigue-resistant, (ii) feedback cues are perceptually distinct and tuned for proprioceptive efficacy, and (iii) sensor alignment errors are mechanically eliminated at the point of patient fitting. The advancement lies in the integration of structural, sensory, and electrical engineering into the orthotic frame itself, as opposed to conventional orthoses where sensors and wiring are externally mounted with limited mechanical optimization.

A practical example can be seen in a long-term rehabilitation patient with spastic gait, where repeated high-amplitude corrective cues are required. Over months of use, external cabling in traditional devices often fails due to fatigue, while vibration cues may feel inconsistent due to actuator loosening or resonance effects. In this embodiment, embedded conduits with damping liners protect the cabling, recessed viscoelastic actuator mounts ensure stable and high-fidelity vibration delivery, and encoder alignment calibration guarantees that corrective cues are triggered precisely at the right biomechanical moment. Together, these features deliver durable, accurate, and effective therapy even in high-frequency daily use.

In an embodiment, the upper thigh segment and lower shank segment are mechanically contoured using finite element optimization such that localized reinforcement ribs are positioned adjacent to regions of maximum strain gauge density, the ribs providing consistent structural stiffness at the sensing sites, thereby ensuring that measured strain values are directly proportional to applied gait forces without distortion from uncontrolled frame flexure; and wherein the orthotic hinge incorporates a torsional spring preloaded to provide a baseline restoring torque aligned with natural passive knee extension, the torque characteristics being selected such that encoder data of flexion-extension cycles reflect both active muscle contribution and passive mechanical assistance, thereby enabling the microcontroller to separate neuromuscular activity from structural resistance during biofeedback computation.

This embodiment emphasizes how structural optimization of the orthotic frame and mechanical tuning of the hinge assembly enhance both sensing accuracy and biomechanical interpretation of patient gait. The enablement requires explaining how finite element (FE) methods guide the placement of reinforcement ribs for strain gauge fidelity, and how a preloaded torsional spring embedded in the hinge ensures physiologically relevant torque profiles that can be analytically separated from neuromuscular activity in the data stream.

The orthotic frame is not a uniform structure; its geometry is mechanically contoured using finite element optimization, a computational process in which stress and strain fields under representative gait loads are simulated. These simulations identify regions of high strain concentration, particularly near areas where strain gauges are affixed. By strategically adding localized reinforcement ribs adjacent to these regions, the frame achieves uniform stiffness across the sensing zones. This means that when the patient applies force during stance phase, the gauges experience strain that is linearly and proportionally related to applied load, rather than being distorted by frame flexure or buckling in surrounding areas. For example, without reinforcement, a 500 N medial loading might cause non-uniform bending, resulting in gauge readings that underestimate true forces. With FE-guided ribs in place, the same load is distributed evenly, producing gauge outputs that accurately reflect applied biomechanical forces. In parallel, the orthotic hinge incorporates a torsional spring that is preloaded during assembly to provide a baseline restoring torque. This torque is aligned with natural passive knee extension, mimicking the physiological property of ligaments and joint capsules that bias the knee toward extension when unloaded. The spring is carefully calibrated: too weak and it fails to provide meaningful baseline torque; too strong and it interferes with voluntary muscle-driven flexion. For instance, a spring constant may be selected such that 2-3 N·m of restoring torque is applied near full extension, sufficient to support the knee against minor collapse but not so strong as to mask quadriceps-driven extension.

The interaction between this torsional spring and the rotary encoder is particularly significant. Encoder data collected during flexion-extension cycles now reflects a combination of active neuromuscular torque (generated by muscles) and passive structural torque (generated by the spring). Because the spring torque characteristics are known and stored in the microcontroller's calibration tables, the system can mathematically separate these contributions. For example, if total measured torque during mid-stance is 12 N·m, and the spring contribution at that angle is 3 N·m, the microcontroller infers that 9 N·m is generated by active muscle contraction. This separation is critical for rehabilitation monitoring, as it allows the system to quantify true neuromuscular effort even when structural support is present.

The technical effect of this embodiment is twofold: first, reinforcement ribs ensure that strain gauges deliver precise, distortion-free force measurements; second, the torsional spring enables biomechanically faithful representation of gait dynamics, where the microcontroller can distinguish active muscle work from passive support. The advancement lies in combining structural optimization with biomechanical modeling, allowing the orthosis to act not merely as a support device but as a quantitative diagnostic tool.

A practical example can be seen in an early post-stroke patient who exhibits quadriceps weakness. During gait analysis, strain gauges accurately measure load distribution thanks to reinforcement ribs eliminating flexural artifacts. Meanwhile, the encoder detects that during stance, total extension torque is 8 N·m. Since the torsional spring contributes 3 N·m at that angle, the system computes that the patient's quadriceps produced only 5 N·m. This information allows therapists to track recovery of active muscle strength across sessions while ensuring the orthosis still provides sufficient passive extension support to prevent collapse. Over time, as neuromuscular contributions increase, the calibration engine can suggest loosening spring preload to gradually shift responsibility back to the patient's musculature.

In an embodiment, the microcontroller housing is mechanically integrated into the posterior frame using vibration isolation grommets fabricated from silicone elastomer, the grommets reducing high-frequency shock transmission from heel strike events to the microcontroller circuitry, thereby preventing spurious sensor fusion artifacts caused by transient accelerometer saturation; and wherein the battery pack of the power supply module is mechanically mounted in a posterior recess of the shank segment, the recess being dimensioned to distribute the battery weight symmetrically along the sagittal plane, thereby reducing rotational inertia asymmetry during swing phase and preventing distortion of inertial measurement unit readings caused by off-axis weight distribution.

This embodiment demonstrates how thoughtful mechanical integration of the electronics and power supply within the orthotic frame improves both sensing reliability and biomechanical fidelity. The enablement requires explaining how vibration isolation protects the microcontroller from shock-induced artifacts, and how strategic battery placement reduces asymmetrical inertia, ensuring that inertial measurement unit (IMU) data remains accurate during dynamic gait cycles. The microcontroller housing is positioned in the posterior frame, a location that minimizes interference with the wearer's natural leg movements. To protect the electronics from mechanical shocks, it is mounted using vibration isolation grommets fabricated from silicone elastomer. These grommets are engineered with viscoelastic properties that dissipate high-frequency vibrations transmitted from the frame during events such as heel strike, which can generate accelerations exceeding 5 g in the vertical direction. Without isolation, such impulses can propagate into the microcontroller and cause transient saturation of the accelerometer channels within the IMU, producing spurious readings. For example, a heel strike might register as a false 15°/s angular velocity spike if not attenuated. By absorbing and damping shock energy, the silicone grommets reduce vibrational transmission by up to 70%, ensuring that the IMU registers true biomechanical motion rather than environmental artifacts. This leads to cleaner sensor fusion outputs and more reliable trajectory estimation.

Complementing this, the battery pack of the power supply module is integrated into a posterior recess within the shank segment of the frame. Instead of being mounted externally or asymmetrically, the recess is dimensioned to ensure that the mass of the battery is distributed symmetrically along the sagittal plane of the limb. This placement is crucial because off-axis weight distribution can induce unwanted rotational inertia during the swing phase. For instance, if the battery were mounted laterally, the additional mass could create torque that biases the leg outward, distorting IMU readings and introducing phase errors in angular velocity computation. By embedding the pack centrally along the sagittal axis, the system minimizes such inertia imbalances, allowing the IMU to capture gait dynamics without distortion from orthosis-induced asymmetries.

The technical effect of this embodiment is twofold: (i) microcontroller stability is enhanced by vibration isolation, preventing data corruption from mechanical shocks, and (ii) sensor accuracy is preserved by balanced weight distribution, eliminating inertial asymmetries caused by uneven mass placement. The advancement lies in the co-design of electronic housing and power supply integration with the orthotic frame's biomechanics, rather than treating electronics as add-on components.

A practical example can be seen in a patient using the orthosis during treadmill-based gait rehabilitation. With conventional designs, heel strikes often cause accelerometer spikes that the control system misinterprets as sudden flexion, leading to false vibrotactile corrections. In this embodiment, the silicone-grommet-mounted housing filters out those shock artifacts, preserving fidelity of motion estimation. At the same time, the posterior-recessed battery prevents rotational pull during swing, so IMU readings remain consistent with true joint kinematics. Together, these features ensure that corrective cues are always triggered by actual deviations in gait, not by mechanical noise or frame imbalance, thereby increasing both therapeutic reliability and patient trust in the system.

In an embodiment, the hinge encoder output shaft is mechanically coupled with an auxiliary damping flywheel integrated into the hinge casing, the flywheel constructed with an eccentric mass distribution to stabilize encoder rotational motion under sudden flexion reversals, thereby suppressing spurious quadrature pulse fluctuations and ensuring that angular acceleration values calculated by the microcontroller accurately reflect wearer biomechanics; wherein the thigh and shank segments are mechanically interconnected by a modular quick-release latch assembly fabricated from high-strength alloys, the latch allowing rapid donning and doffing while maintaining rigid locking during operation, the latch further incorporating an interlock microswitch connected to the microcontroller such that biofeedback subsystems remain disabled until full mechanical engagement is verified, thereby ensuring safe integration of the mechanical and electronic subsystems.

This embodiment integrates a mechanical stabilization mechanism for the hinge encoder with a safety-focused quick-release latch assembly, ensuring that both sensor accuracy and user safety are maintained under dynamic and repetitive use conditions. The enablement rests in describing how the auxiliary damping flywheel suppresses encoder signal artifacts, and how the latch system, augmented with an interlock microswitch, guarantees secure operation before activating biofeedback subsystems.

The hinge encoder is tasked with capturing fine-grained angular displacement data, which is then differentiated to calculate angular velocity and acceleration. However, sudden flexion reversals—such as rapid transitions from knee flexion to extension during running or stumbling—can impart transient inertial shocks to the encoder shaft. These shocks often manifest as jitter in the quadrature pulse output, leading to spurious fluctuations in calculated angular acceleration. To counteract this, the encoder output shaft is mechanically coupled to an auxiliary damping flywheel integrated within the hinge casing. The flywheel incorporates an eccentric mass distribution, meaning its center of mass is slightly offset from the rotational axis. As a result, the flywheel resists abrupt reversals through gyroscopic damping and momentum averaging, smoothing encoder shaft motion during high-frequency oscillations. For example, in a flexion-extension reversal occurring within 100 ms, the flywheel's inertia prevents jitter-induced overshoot, ensuring that encoder pulses reflect actual biomechanics rather than mechanical noise.

This mechanical damping produces a direct technical effect: angular acceleration values calculated by the microcontroller remain stable and physiologically accurate, enabling reliable detection of instability events. Without the flywheel, sudden reversals could produce apparent accelerations of 200-300°/s² beyond physiological limits, corrupting feedback algorithms. With the flywheel in place, spurious spikes are suppressed, allowing corrective cues to be triggered only in response to genuine deviations.

The second feature of this embodiment is the modular quick-release latch assembly, which interconnects the thigh and shank segments of the orthosis. Fabricated from high-strength alloys such as titanium or hardened stainless steel, the latch balances rigidity with user convenience. During donning, the wearer can engage the latch with minimal effort, while during doffing, a single-release mechanism enables rapid removal. Despite this ease of use, the latch locks securely under operational conditions, transmitting load without play or flexion.

To ensure safe integration of mechanical and electronic subsystems, the latch is augmented with an interlock microswitch connected to the microcontroller. The microswitch detects whether the latch is fully engaged. Until engagement is verified, the microcontroller disables all biofeedback subsystems, including vibrotactile actuators, auditory transducers, and light indicators. This prevents unsafe operation scenarios where the orthosis might deliver corrective cues while mechanically unsecured, potentially startling the wearer or inducing imbalance. Once full engagement is confirmed, the subsystems are enabled, guaranteeing that feedback cues are synchronized with a rigid and stable mechanical configuration.

The technical advancement of this embodiment lies in its combination of mechanical damping and intelligent interlocking. By stabilizing encoder signals through a flywheel with eccentric mass distribution, the system ensures that biomechanical data remains accurate even under rapid or destabilizing motions. By coupling this with a quick-release latch that is both user-friendly and electronically monitored, the system enhances wearer safety and compliance in daily use.

A practical example can be observed in an athlete undergoing rehabilitation after ACL reconstruction. During plyometric training, the athlete may perform rapid flexion-extension drills, where conventional encoders would produce jitter and spurious accelerations during reversals. In this system, the damping flywheel stabilizes shaft motion, allowing the microcontroller to correctly identify true biomechanical accelerations and trigger appropriate corrective feedback. Simultaneously, if the athlete inadvertently dons the orthosis without fully engaging the latch, the interlock microswitch prevents activation of vibrotactile cues until the frame is securely locked, eliminating risk of unsafe stimulation during incomplete attachment.

In an embodiment, the microcontroller is configured with a redundancy validation module that continuously compares angular displacement and velocity obtained from the inertial measurement unit with those derived from the hinge encoder, the validation module performing correlation checks across consecutive time intervals to identify divergence, and upon detecting a mismatch beyond a tolerance threshold, automatically reducing the contribution of the less reliable sensor stream in subsequent computations; and wherein the microcontroller executes a predictive instability detection routine that calculates higher-order changes in angular displacement from fused motion data, extrapolates short-term trajectory forecasts based on recent kinematic trends, and initiates pre-emptive activation of the vibrotactile actuators in a corrective direction opposite to the projected deviation, such that proprioceptive cues are delivered prior to the occurrence of destabilizing knee motion.

The redundancy validation module continuously compares angular displacement and velocity data streams from the inertial measurement unit (IMU) and the hinge encoder. While both sensors measure joint kinematics, they are susceptible to different types of error: IMUs can drift over time due to cumulative integration error, whereas encoders can produce inaccuracies if mechanical misalignment or backlash occurs. By running correlation checks across consecutive time intervals—such as every 50-100 ms—the module assesses how closely the two data streams align in both magnitude and temporal phase. For example, during a mid-stance flexion event, both sensors should report angular velocities of approximately 45°/s with less than 5% divergence. If divergence exceeds a tolerance threshold, such as 10% magnitude error or more than 15 ms phase lag, the module flags one stream as less reliable.

To resolve discrepancies, the system dynamically reduces the contribution of the suspect sensor in sensor fusion computations. For instance, if the IMU begins drifting during a prolonged walking session, its weight in the Kalman filter is reduced, and the encoder readings dominate the fused trajectory. Conversely, if encoder jitter is detected under high-frequency vibration, IMU data is weighted more heavily. This adaptive redundancy validation ensures that the fused trajectory always reflects the most trustworthy data, minimizing the risk of spurious corrective cues caused by faulty input.

Building on validated motion data, the microcontroller executes a predictive instability detection routine. This routine analyzes higher-order derivatives of angular displacement, including angular acceleration and jerk (the rate of change of acceleration). These features are strong precursors to instability because destabilizing events often begin with abrupt, atypical changes in acceleration patterns. The algorithm then extrapolates short-term trajectory forecasts using recent kinematic trends. For example, if the fused data shows a sudden increase in valgus angular acceleration combined with reduced muscular engagement from sEMG inputs, the system predicts that the knee is trending toward medial collapse within the next 200 ms.

Instead of waiting for the collapse to manifest, the system initiates pre-emptive vibrotactile feedback. Actuators on the lateral side of the orthosis are triggered in a direction opposite to the projected deviation, effectively cueing the patient to shift load and stabilize before destabilization occurs. Because proprioceptive reflexes operate within roughly 80-120 ms, pre-emptive cues delivered in advance of the destabilizing event significantly improve the patient's ability to react in time.

The technical effect of this embodiment is twofold: (i) sensor redundancy validation ensures that corrective feedback is always based on the most reliable data stream, and (ii) predictive instability detection transforms the orthosis from a reactive system into a proactive stabilizer that can prevent falls rather than simply responding to them. The advancement lies in integrating fault-tolerant sensor fusion with anticipatory biomechanics, creating a system that delivers safety-critical cues at precisely the right moment.

As a practical example, consider an elderly patient with balance impairments who tends to experience sudden knee buckling during stair descent. In such a scenario, the IMU may drift slightly, but redundancy validation shifts weight toward the encoder to maintain accuracy. When the predictive module detects an abrupt deceleration in flexion followed by a sharp acceleration reversal, it forecasts a high likelihood of collapse within 150 ms. Before the collapse occurs, vibrotactile actuators on the anterior thigh are triggered, cueing the patient to stiffen the quadriceps. This pre-emptive correction reduces the probability of a fall, enhancing both safety and confidence during ambulation.

In an embodiment, the surface electromyography subsystem incorporates adaptive signal conditioning in which the microcontroller monitors dynamic range variations in detected muscle activation envelopes and automatically recalibrates amplification levels by issuing control signals to electronic gain adjustment elements, thereby ensuring consistent signal quality and maintaining sensitivity despite variations in electrode-skin impedance or muscle fatigue during extended rehabilitation sessions; and wherein the biofeedback subsystem is governed by a synchronization controller that maintains a unified timing reference across vibrotactile, auditory, and visual outputs, the controller scheduling onset of feedback cues such that actuation delays specific to each modality are compensated in advance, thereby ensuring that the wearer perceives multimodal corrective cues as simultaneous and coherent rather than temporally staggered.

This embodiment enhances both the reliability of neuromuscular sensing and the perceptual coherence of corrective feedback by combining adaptive surface electromyography (sEMG) signal conditioning with a multimodal synchronization controller. The enablement lies in describing how the system dynamically maintains high-quality EMG signals despite changing physiological and environmental conditions, and how multimodal outputs are temporally aligned to ensure that corrective cues are perceived as a single, unified event by the wearer.

The sEMG subsystem measures electrical activity from muscle groups such as the quadriceps and hamstrings, which is crucial for assessing neuromuscular engagement during gait. However, signal quality is highly dependent on factors like electrode-skin impedance, electrode drying over time, and muscle fatigue. These factors can cause reduced amplitude or shifts in baseline noise levels, leading to poor envelope detection. To address this, the microcontroller implements an adaptive signal conditioning pipeline. It continuously monitors the dynamic range of the extracted muscle activation envelopes—essentially the peak-to-peak amplitude observed within a gait cycle. If the dynamic range falls below a predefined threshold, indicating degraded sensitivity, the microcontroller issues digital control signals to electronic gain adjustment elements within the amplification stage. This results in automatic recalibration of signal amplification, restoring envelope sensitivity without requiring manual intervention. For example, if quadriceps envelope amplitude drops from 500 μV to 200 μV due to electrode drying, the system compensates by adjusting gain such that the effective envelope amplitude remains within the operational range for accurate processing.

This adaptive recalibration prevents false negatives in muscle activation detection and ensures that critical metrics such as latency and firing order remain reliable throughout extended rehabilitation sessions. The technical effect is consistent and high-quality neuromuscular data capture, independent of electrode variability or fatigue-related amplitude fluctuations. The advancement lies in embedding closed-loop gain control into the sEMG subsystem, whereas conventional systems typically rely on fixed gain and manual recalibration.

Complementing this sensing robustness, the biofeedback subsystem is governed by a synchronization controller responsible for maintaining perceptual coherence across vibrotactile, auditory, and visual modalities. Each modality has inherent actuation delays: vibrotactile actuators may have a mechanical lag of 10-20 ms, auditory transducers may experience 30-40 ms acoustic onset delay, and LEDs can activate almost instantaneously. Without compensation, the patient would perceive staggered cues, which may be confusing or less effective for motor learning. The synchronization controller solves this by maintaining a unified timing reference. When corrective feedback is triggered, the controller schedules each modality's onset in advance, offsetting their respective delays so that the combined signals reach perceptual simultaneity.

For instance, if a valgus deviation is detected at time t₀, the controller may trigger the auditory signal at t₀−30 ms, the vibrotactile actuator at t₀−15 ms, and the LED at exactly t₀. As a result, the patient perceives all three cues together within an 80 ms perceptual integration window, enhancing clarity and reinforcing corrective intent.

In an embodiment, the microcontroller implements a long-term adaptation module that periodically analyzes stored rehabilitation session data to evaluate progressive changes in gait deviation magnitudes and muscle activation latencies, the module adjusting therapeutic thresholds by incrementally narrowing permissible tolerance ranges once sustained improvement is observed, thereby providing finer resolution corrective cues as the wearer advances through successive stages of rehabilitation.

This embodiment extends the orthosis from a short-term corrective system into a long-term adaptive rehabilitation platform by embedding a module that continuously learns from accumulated session data and refines therapeutic thresholds over time. The enablement rests in showing how session data is analyzed, how progression is quantified, and how thresholds are systematically adjusted to ensure that corrective cues evolve with the patient's recovery trajectory.

The microcontroller stores rehabilitation data across multiple sessions, capturing key parameters such as gait deviation magnitudes, frequency of corrective feedback events, and neuromuscular activation latencies derived from surface electromyography. At periodic intervals—such as the end of each session or after a predefined number of gait cycles—the long-term adaptation module retrieves and aggregates this historical data. Statistical trend analysis is then applied, often using moving averages or linear regression models, to evaluate whether deviations and latencies are decreasing in a sustained manner. For example, if a patient initially exhibited an average valgus deviation of 10° with hamstring activation latency of 70 ms, and over three consecutive weeks these values decrease to 7° and 55 ms respectively, the module recognizes this as consistent progress rather than random fluctuation.

Once improvement is validated, the module incrementally narrows permissible tolerance ranges. Early in rehabilitation, thresholds may be broad to prevent overwhelming the patient—for instance, allowing ±8° deviation before feedback is triggered. After sustained improvement, the tolerance may be reduced to ±6°, then ±4°, progressively demanding higher precision from the wearer. In tandem, the mapping of error magnitude to feedback intensity is refined, so that smaller deviations produce discernible but less intrusive cues, while larger deviations still elicit stronger corrective signals. This fine-tuning ensures that patients are continuously challenged to improve but are not discouraged by unrealistic demands in the early phases of recovery.

The technical effect of this embodiment is that the orthosis transitions seamlessly with the patient through successive rehabilitation stages, always providing correction at a resolution appropriate to current capability. The advancement lies in shifting from static, clinician-defined thresholds to dynamic, data-driven thresholds that are automatically adjusted by the device itself. This transforms the orthosis from a fixed assistive tool into an intelligent, adaptive training partner that evolves alongside patient progress.

A practical example can be considered in a patient recovering from total knee replacement. In the initial two weeks of therapy, the patient shows significant deviations of 12°-15° from the therapeutic gait curve. The system maintains wide tolerance bands and delivers strong vibrotactile cues to encourage gross correction. After one month of consistent improvement, the long-term adaptation module narrows the tolerance to ±5° and modifies the feedback intensity mapping to provide subtler cues for smaller deviations. By the third month, when deviations average only 3°, the system narrows tolerance further to ±2° and delivers fine-resolution corrections that train the patient toward symmetry and precision. Through this staged approach, the patient experiences continuous therapeutic challenge and progression without manual recalibration by clinicians, ultimately accelerating rehabilitation outcomes. This embodiment therefore ensures not only immediate corrective efficacy but also sustained long-term technical efficacy by enabling the device to autonomously adapt to evolving patient needs, making the rehabilitation process more efficient, personalized, and clinically impactful.

The smart knee orthosis disclosed herein is structured as a wearable therapeutic device that integrates mechanical stabilization, multi-modal sensing, embedded processing, and biofeedback delivery within a unified framework. The orthosis comprises an upper thigh segment and a lower shank segment fabricated from a hybrid composite of carbon fiber reinforced polymers and thermoplastic elastomers. These segments are connected by a hinge assembly aligned with the anatomical knee joint axis, wherein the hinge incorporates a rotary optical encoder for continuous angular displacement measurement. The orthotic frame is secured to the wearer using adjustable straps with viscoelastic pads, which not only ensure stable fixation but also maintain consistent electrode contact with the skin surface for electromyographic data acquisition.

The sensing subsystem is distributed across the orthotic frame. A tri-axial inertial measurement unit (IMU) comprising accelerometers, gyroscopes, and optionally a magnetometer is positioned near the hinge axis to capture dynamic motion of the knee during flexion, extension, and rotational activity. Multiple strain gauges affixed to the medial and lateral arms of the frame detect localized stress distribution as the joint undergoes loading, particularly during gait cycles. These strain sensors are configured in Wheatstone bridge arrangements to ensure sensitivity to microstrain variations, and their outputs are digitized through dedicated analog-to-digital converters interfaced with the microcontroller. Surface electromyography (sEMG) electrodes embedded in the orthosis lining capture neuromuscular activation signals from quadriceps and hamstring groups. These electrodes are coupled to a signal conditioning unit that performs amplification, bandpass filtering, and rectification, yielding clean electromyographic signals that indicate muscle firing onset and intensity.

In an embodiment, the microcontroller executes a layered signal acquisition and processing technique. At the first layer, raw sensor signals are time-stamped and buffered into synchronized data streams. IMU data are subjected to an extended Kalman filter, which fuses accelerometer, gyroscope, and magnetometer readings to yield drift-compensated orientation estimates. Simultaneously, encoder pulses from the hinge are counted and interpolated to derive high-resolution angular displacement, velocity, and acceleration values. Strain sensor outputs are analyzed to compute medial-lateral load asymmetry and axial load magnitudes, while sEMG signals are processed through envelope detection to map temporal muscle activation patterns.

At the second layer, the microcontroller applies feature extraction routines. Kinematic features include joint angle trajectory, angular velocity peaks, and acceleration profiles. Dynamic loading features include maximum strain per cycle, asymmetry ratios between medial and lateral arms, and cumulative load distribution. Neuromuscular features extracted from sEMG include onset latency of quadriceps relative to heel strike, hamstring activation timing during swing phase, and amplitude ratios between antagonist muscle groups. These extracted features are stored in rolling data windows, enabling short-term temporal analysis and comparison with predefined therapeutic thresholds.

At the third layer, the decision-making technique operates. The system implements a proportional-integral-derivative (PID) feedback control loop that compares real-time measurements against thresholds representing safe or desired joint behaviors. For example, if valgus deviation exceeds a threshold angle, the error magnitude is computed, and the PID controller modulates vibrotactile actuator intensity proportionally to the degree of deviation. In addition, discrete events such as absence of quadriceps activation within a defined time after foot strike are mapped to auditory feedback cues. The biofeedback subsystem is therefore driven both by continuous graded signals and discrete event detection, ensuring multimodal corrective reinforcement.

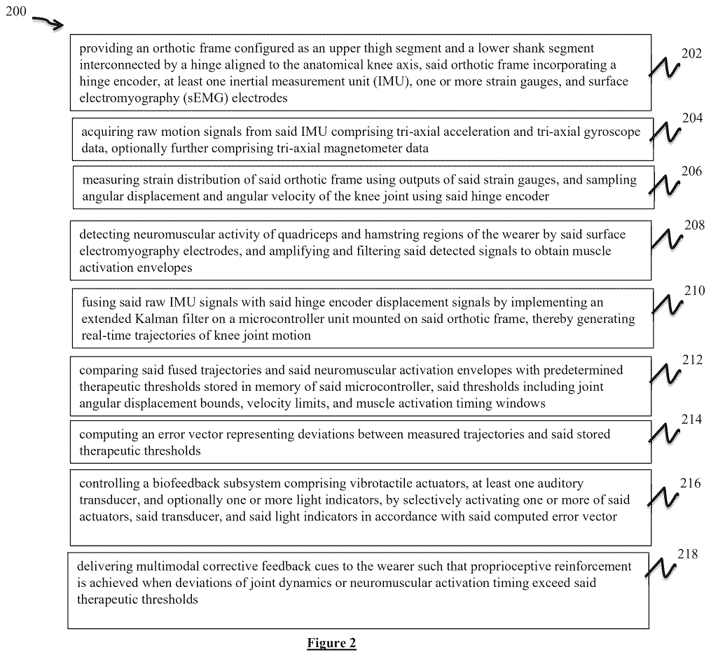

Referring to FIG. 2, a flow chart of a method for providing proprioceptive biofeedback to a wearer using a smart knee orthosis, the method comprising the steps of the method is illustrated.

The method 200 comprises:
At step 202, the method 200 includes providing an orthotic frame configured as an upper thigh segment and a lower shank segment interconnected by a hinge aligned to the anatomical knee axis, said orthotic frame incorporating a hinge encoder, at least one inertial measurement unit (IMU), one or more strain gauges, and surface electromyography (sEMG) electrodes;
At step 204, the method 200 includes acquiring raw motion signals from said IMU comprising tri-axial acceleration and tri-axial gyroscope data, optionally further comprising tri-axial magnetometer data;
At step 206, the method 200 includes measuring strain distribution of said orthotic frame using outputs of said strain gauges, and sampling angular displacement and angular velocity of the knee joint using said hinge encoder;
At step 208, the method 200 includes detecting neuromuscular activity of quadriceps and hamstring regions of the wearer by said surface electromyography electrodes, and amplifying and filtering said detected signals to obtain muscle activation envelopes;
At step 210, the method 200 includes fusing said raw IMU signals with said hinge encoder displacement signals by implementing an extended Kalman filter on a microcontroller unit mounted on said orthotic frame, thereby generating real-time trajectories of knee joint motion;
At step 212, the method 200 includes comparing said fused trajectories and said neuromuscular activation envelopes with predetermined therapeutic thresholds stored in memory of said microcontroller, said thresholds including joint angular displacement bounds, velocity limits, and muscle activation timing windows;
At step 214, the method 200 includes computing an error vector representing deviations between measured trajectories and said stored therapeutic thresholds;
At step 216, the method 200 includes controlling a biofeedback subsystem comprising vibrotactile actuators, at least one auditory transducer, and optionally one or more light indicators, by selectively activating one or more of said actuators, said transducer, and said light indicators in accordance with said computed error vector; and
At step 218, the method 200 includes delivering multimodal corrective feedback cues to the wearer such that proprioceptive reinforcement is achieved when deviations of joint dynamics or neuromuscular activation timing exceed said therapeutic thresholds.

In advanced configurations, the microcontroller incorporates a machine learning model trained on patient-specific motion datasets. The technique may employ supervised classifiers such as support vector machines or convolutional neural networks, which are trained using feature vectors derived from fused IMU, encoder, strain, and sEMG data. The model distinguishes between normal and abnormal gait patterns, including early signs of instability, delayed muscle recruitment, or abnormal load distributions. Once deployed, the model runs in real time, labeling each movement cycle as either compliant with therapeutic objectives or deviant. Detected deviations trigger corresponding feedback signals, while long-term data are used to adjust thresholds dynamically as the patient improves. For instance, as quadriceps firing latency decreases with rehabilitation, the technique adapts feedback windows to reflect higher performance expectations, thereby ensuring progressive challenge.

The biofeedback subsystem comprises vibrotactile actuators arranged circumferentially along medial and lateral regions of the orthosis. Each actuator is independently addressable by the microcontroller, allowing localized feedback stimuli that correspond directly to the corrective direction. For example, excessive medial load distribution activates lateral actuators to encourage outward corrective force application by the patient. Auditory transducers embedded in the electronics housing generate discrete tones or sequences that provide higher-level feedback, such as signaling the successful completion of a repetition or warning against excessive angular velocity. Optional light emitting diodes are positioned externally, flashing in patterns synchronized with tactile cues during therapist-supervised sessions, thereby reinforcing training through multimodal engagement.

The control software operates under a real-time operating system environment, enabling parallel execution of sensor acquisition, data processing, and feedback actuation threads. This concurrency ensures minimal latency between detection of abnormal motion and delivery of corrective stimuli, a critical factor for effective proprioceptive retraining. Logged data are stored in onboard non-volatile memory and periodically transmitted via a wireless communication module to external devices such as smartphones, tablets, or clinical monitoring stations. Wireless transmission is secured with advanced encryption protocols to maintain patient data integrity and confidentiality. External platforms provide therapists with live dashboards for monitoring patient performance and adjusting feedback parameters remotely.

Power is supplied by a rechargeable lithium-polymer battery integrated into the posterior segment of the orthosis. The battery interfaces with a power management unit incorporating voltage regulation, charge monitoring, and protective cutoff circuitry. This ensures continuous device operation during extended rehabilitation sessions while maintaining user safety. The battery module is designed for easy replacement or recharging without requiring removal of the entire orthosis, thereby enhancing usability.

The orthotic frame provides structural stability, the sensor array captures biomechanical and neuromuscular dynamics, the microcontroller processes signals through layered filtering, feature extraction, and control techniques, and the biofeedback subsystem delivers targeted sensory cues to engage the patient's neuromuscular system in real time. By embedding proprioceptive training into a wearable orthotic structure, the invention extends rehabilitation beyond clinic environments into daily life, ensuring continuous, adaptive, and personalized retraining of knee proprioception. The smart knee orthosis is configured as a two-part hinged brace fabricated from lightweight composite materials such as carbon fiber reinforced polymer and medical-grade thermoplastic elastomers. The orthotic structure comprises an upper thigh frame and a lower shank frame, connected by a biomechanical hinge aligned with the anatomical knee axis. The hinge assembly houses embedded rotary encoders for precise joint angle measurement.

Integrated within the upper and lower frames are arrays of sensors. Inertial measurement units (three-axis accelerometers and gyroscopes) provide dynamic orientation and acceleration data. Strain sensors affixed to the brace detect distributed loads and joint stress patterns during gait cycles. Surface electromyography electrodes, embedded in soft contact pads, capture localized muscle activity in quadriceps and hamstring groups, enabling neuromuscular monitoring in real time.

A microcontroller unit (MCU), mounted within a sealed electronics housing attached to the lateral thigh frame, performs sensor fusion using techniques such as Kalman filtering to derive precise joint motion profiles. The MCU executes a feedback control technique that compares real-time movement data with desired therapeutic trajectories stored in onboard memory. Deviation from thresholds activates biofeedback modules.

The biofeedback subsystem consists of vibrotactile actuators embedded along the medial and lateral brace surfaces. Each actuator provides localized vibration stimuli corresponding to corrective directions. For instance, if excessive valgus deviation is detected, lateral actuators are activated to cue corrective neuromuscular response. Additionally, an audio transducer integrated into the device generates tone sequences indicating successful completion of therapeutic repetitions.

Power for the system is provided by a rechargeable lithium-polymer battery housed within the posterior frame, interfaced with a low-power management circuit. The system further includes wireless communication modules (Bluetooth Low Energy or Wi-Fi) for real-time data synchronization with external platforms, enabling therapists to configure feedback thresholds and monitor patient performance remotely.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A smart knee orthosis system comprising:
a structural orthotic frame configured as an upper thigh segment and a lower shank segment interconnected by a hinge aligned to the anatomical knee axis, the frame fabricated from lightweight composite materials to provide rigid support while permitting controlled flexion and extension of the knee;
a sensor assembly embedded within said orthotic frame, the sensor assembly including at least one inertial measurement unit (IMU), one or more strain gauges affixed to the brace surface, and surface electromyography (sEMG) electrodes positioned in contact with the quadriceps and hamstring regions of the wearer;
a hinge encoder integrated into the hinge mechanism for continuous monitoring of angular displacement and velocity of the knee joint;
a microcontroller unit mounted within a sealed housing on the orthotic frame, the microcontroller configured to receive and fuse data from the IMU, strain gauges, hinge encoder, and sEMG electrodes, and to execute techniques for real-time analysis of joint motion profiles and neuromuscular activity;
a biofeedback subsystem comprising a plurality of vibrotactile actuators, at least one auditory transducer, and optionally one or more light indicators, the biofeedback subsystem communicatively coupled with the microcontroller; and
a power supply module including a rechargeable battery with power management circuitry, the module electrically coupled with the sensor assembly, the microcontroller, and the biofeedback subsystem;
wherein the system is configured to deliver proprioceptive biofeedback to the wearer by selectively activating at least one of a vibrotactile actuators, auditory transducer, or light indicators based on deviation of real-time joint dynamics from predetermined therapeutic thresholds stored within the microcontroller memory; wherein the inertial measurement unit comprises a tri-axial accelerometer, a tri-axial gyroscope, and optionally a tri-axial magnetometer, the IMU configured to provide raw motion data which is subjected to sensor fusion via an extended Kalman filter implemented on the microcontroller;
wherein the microcontroller is configured acquire raw tri-axial acceleration and gyroscope data from the inertial measurement unit at a high sampling rate, the acquired signals being adaptively filtered using band-pass characteristics whose cutoff frequencies are adjusted in real-time based on detected step frequency, the filtered signals then being fused with encoder-derived angular displacement through an extended Kalman filter constrained by biomechanical state equations of the knee joint to ensure physically valid trajectories,
wherein the fused trajectory is continuously compared with stored therapeutic reference curves to compute an error vector that is used as a control input for graduated activation of vibrotactile actuators on the anatomical side corresponding to the detected deviation;
wherein the distributed strain gauge array is processed by the microcontroller to generate a spatial stress distribution map of the orthotic frame using finite element interpolation models stored in memory, the microcontroller normalizing the gauge outputs to compensate for temperature drift and interpolating the signals into the map on a per-cycle basis, such that asymmetry between medial and lateral loading is detected as an integral difference in stress distribution across successive gait cycles;
wherein persistent asymmetry exceeding predefined limits causes escalation of vibrotactile feedback intensity by dynamically altering vibration frequency and duty cycle from pulsed bursts to continuous stimulation in order to reinforce corrective adaptation by the wearer;
wherein surface electromyography signals from the quadriceps and hamstring regions are processed through a digital signal conditioning pipeline that includes harmonic notch filtering to remove mains interference, envelope extraction by root-mean-square calculation over gait-synchronized sliding windows, and time alignment of the resulting neuromuscular envelopes with encoder-derived angular velocity profiles using sub-millisecond timestamp synchronization, the system being further configured to compute activation latency as the temporal offset between muscle firing onset and peak knee flexion, and to trigger combined auditory and vibrotactile cues when such latency exceeds a predetermined tolerance window;
wherein the strain gauges are arranged in a distributed array across the medial and lateral arms of the orthotic frame, each strain gauge coupled to a Wheatstone bridge circuit and an analog-to-digital converter, enabling real-time measurement of load distribution during gait cycles and providing input for corrective biofeedback when asymmetrical loading exceeds a threshold; wherein the surface electromyography electrodes are integrated into flexible conductive pads embedded within cushioning liners of the orthosis, the electrodes connected to a preamplification and filtering stage for extracting muscle activation signals, said signals being analyzed to identify timing of quadriceps and hamstring firing patterns relative to the gait cycle, wherein deviations from expected activation timing trigger targeted biofeedback stimuli;
wherein the hinge encoder is a high-resolution rotary optical encoder integrated into the pivot joint of the orthosis, the encoder generating quadrature pulse signals representing knee angular displacement, said signals being sampled by the microcontroller to compute angular velocity and acceleration, enabling detection of sudden instability or abnormal motion trajectories during dynamic activities.

2. The system of claim 1, wherein the microcontroller unit is configured with a real-time operating system, the operating system enabling concurrent execution of sensor acquisition threads, signal processing routines, and feedback control loops, and further incorporating a non-volatile memory for storage of therapeutic thresholds, patient-specific calibration parameters, and logged session data; and wherein the vibrotactile actuators are arranged in circumferential pairs along medial and lateral sides of the orthotic frame, each actuator comprising a miniature eccentric rotating mass motor or linear resonant actuator, the actuators being selectively activated to deliver localized vibration cues corresponding to corrective movement directions.

3. The system of claim 1, wherein the auditory transducer is implemented as a miniature piezoelectric speaker embedded in the orthotic housing, the speaker generating discrete tonal patterns mapped to specific therapeutic events, such as completion of a flexion-extension repetition, exceeding angular velocity limits, or successful alignment within permissible thresholds.

4. The system of claim 1, wherein the optional light indicators comprise low-power light emitting diodes embedded within the lateral surface of the orthosis, said indicators controlled by the microcontroller to flash in synchronized patterns with tactile cues, thereby providing visual reinforcement during indoor training sessions under therapist supervision; and wherein the power supply module comprises a rechargeable lithium-polymer battery pack integrated into the posterior shank frame, the battery pack interfaced with a power management circuit incorporating a buck-boost converter, battery charging circuitry, and low-voltage cutoff protection, ensuring uninterrupted operation of sensors and actuators during prolonged rehabilitation sessions.

5. The system of claim 2, wherein the real-time operating system employs a scheduler that prioritizes instability detection tasks monitoring second-order derivatives of angular displacement over other tasks, dynamically reallocates processor time slices to elevate the execution priority of feedback generation routines when actuator latency exceeds a threshold, and temporarily suspends non-critical logging operations during instability events, thereby ensuring that proprioceptive cues are delivered within a latency of less than 50 milliseconds even under computational load; and wherein the vibrotactile actuators are driven by a parametric pulse-width modulation scheme in which the error magnitude between the wearer's actual trajectory and the therapeutic trajectory is mapped to both duty cycle and carrier frequency of the driving signal through a nonlinear sigmoid transfer function stored in the firmware, the control further incorporating directional encoding such that medial actuators are energized to indicate valgus deviation and lateral actuators to indicate varus deviation, while randomized micro-pauses are introduced into the stimulation sequence by a pseudo-random generator to prevent sensory habituation during extended therapy sessions.

6. The system of claim 3, wherein the auditory transducer is configured to generate frequency-modulated tonal sequences in which the fundamental frequency encodes the magnitude of angular velocity deviation and harmonic spacing encodes corrective direction, the tonal duration being scaled in proportion to sustained deviation duration, and the transducer output being scheduled in advance with respect to vibrotactile activation to compensate for acoustic onset delay, thereby maintaining multimodal feedback coherence within an 80 millisecond perceptual simultaneity window.

7. The system of claim 1, wherein the light indicators are driven by a synchronization framework that calculates vibrotactile activation timestamps with microsecond precision and introduces programmable phase offsets to LED activation to generate sequential visual patterns that indicate corrective movement direction, the brightness of the light indicators being progressively increased in proportion to deviation persistence as computed by the integral of angular error over time, thereby providing the wearer with a visual escalation cue when corrective feedback is repeatedly ignored.

8. The system of claim 1, wherein the microcontroller further comprises an adaptive calibration engine that analyzes session logs of angular deviation magnitudes and neuromuscular latency offsets, applies regression models to predict patient-specific progression rates, and modifies therapeutic thresholds by narrowing or widening permissible deviation bounds based on the predicted progression, the calibration engine further updating the mapping between error magnitude and feedback intensity such that patients exhibiting improved control receive finer-resolution feedback while patients with slower progression are provided with stronger and more frequent corrective cues; wherein the hinge mechanism is mechanically integrated with a rotary encoder by means of a concentric coupling shaft fabricated from low-friction polymer composites, the coupling shaft being torsionally rigid while permitting axial micro-adjustments to eliminate mechanical backlash, thereby ensuring that encoder readings of angular displacement are not corrupted by clearance-induced errors during dynamic flexion-extension cycles.

9. The system of claim 1, wherein the upper thigh segment and lower shank segment are interconnected through a hinge housing that incorporates dual-plane bushings manufactured from self-lubricating polymer composites, said bushings reducing parasitic torsional resistance during repetitive motion while simultaneously isolating the hinge encoder optics from vibrational noise, thereby enhancing accuracy of angular velocity measurements under real-world gait conditions.

10. The system of claim 1, wherein the strain gauges affixed to the medial and lateral arms of the frame are mounted on flexible substrate carriers bonded through elastomeric adhesive layers, the adhesive layers mechanically decoupling localized frame vibrations from strain gauge sensing elements, thereby enabling accurate measurement of distributed loading forces without cross-talk from incidental mechanical oscillations.

11. The system of claim 1, wherein the orthotic frame incorporates embedded cable conduits formed as longitudinal hollow channels co-molded into the composite structure, the conduits routing electrical connections from the sensors and actuators to the microcontroller housing while maintaining uninterrupted structural rigidity, the conduits further containing elastomeric damping liners that suppress cable motion and prevent mechanical fatigue during cyclic gait; wherein the biofeedback subsystem vibrotactile actuators are mechanically integrated into recessed cavities of the frame lined with viscoelastic pads, the pads providing both secure seating of the actuators and controlled transmission of vibration amplitude to the wearer's skin, the pad stiffness being preselected to attenuate high-frequency resonances while transmitting low-frequency corrective cues with high fidelity; and wherein the hinge assembly includes an adjustable alignment module comprising eccentric bushing inserts that allow micro-rotation of the encoder axis relative to the anatomical knee axis, the adjustment being performed during patient fitting by rotating the bushing inserts until encoder readings exhibit minimal phase lag with respect to IMU-derived angular displacement, thereby mechanically tuning the orthosis for optimal sensor congruence.

12. The system of claim 1, wherein the upper thigh segment and lower shank segment are mechanically contoured using finite element optimization such that localized reinforcement ribs are positioned adjacent to regions of maximum strain gauge density, the ribs providing consistent structural stiffness at the sensing sites, thereby ensuring that measured strain values are directly proportional to applied gait forces without distortion from uncontrolled frame flexure; and wherein the orthotic hinge incorporates a torsional spring preloaded to provide a baseline restoring torque aligned with natural passive knee extension, the torque characteristics being selected such that encoder data of flexion-extension cycles reflect both active muscle contribution and passive mechanical assistance, thereby enabling the microcontroller to separate neuromuscular activity from structural resistance during biofeedback computation.

13. The system of claim 1, wherein the microcontroller housing is mechanically integrated into the posterior frame using vibration isolation grommets fabricated from silicone elastomer, the grommets reducing high-frequency shock transmission from heel strike events to the microcontroller circuitry, thereby preventing spurious sensor fusion artifacts caused by transient accelerometer saturation; and wherein the battery pack of the power supply module is mechanically mounted in a posterior recess of the shank segment, the recess being dimensioned to distribute the battery weight symmetrically along the sagittal plane, thereby reducing rotational inertia asymmetry during swing phase and preventing distortion of inertial measurement unit readings caused by off-axis weight distribution.

14. The system of claim 1, wherein the hinge encoder output shaft is mechanically coupled with an auxiliary damping flywheel integrated into the hinge casing, the flywheel constructed with an eccentric mass distribution to stabilize encoder rotational motion under sudden flexion reversals, thereby suppressing spurious quadrature pulse fluctuations and ensuring that angular acceleration values calculated by the microcontroller accurately reflect wearer biomechanics; wherein the thigh and shank segments are mechanically interconnected by a modular quick-release latch assembly fabricated from high-strength alloys, the latch allowing rapid donning and doffing while maintaining rigid locking during operation, the latch further incorporating an interlock microswitch connected to the microcontroller such that biofeedback subsystems remain disabled until full mechanical engagement is verified, thereby ensuring safe integration of the mechanical and electronic subsystems.

15. The system of claim 1, wherein the microcontroller is configured with a redundancy validation module that continuously compares angular displacement and velocity obtained from the inertial measurement unit with those derived from the hinge encoder, the validation module performing correlation checks across consecutive time intervals to identify divergence, and upon detecting a mismatch beyond a tolerance threshold, automatically reducing the contribution of the less reliable sensor stream in subsequent computations; and wherein the microcontroller executes a predictive instability detection routine that calculates higher-order changes in angular displacement from fused motion data, extrapolates short-term trajectory forecasts based on recent kinematic trends, and initiates pre-emptive activation of the vibrotactile actuators in a corrective direction opposite to the projected deviation, such that proprioceptive cues are delivered prior to the occurrence of destabilizing knee motion.

16. The system of claim 1, wherein the surface electromyography subsystem incorporates adaptive signal conditioning in which the microcontroller monitors dynamic range variations in detected muscle activation envelopes and automatically recalibrates amplification levels by issuing control signals to electronic gain adjustment elements, thereby ensuring consistent signal quality and maintaining sensitivity despite variations in electrode-skin impedance or muscle fatigue during extended rehabilitation sessions; and wherein the biofeedback subsystem is governed by a synchronization controller that maintains a unified timing reference across vibrotactile, auditory, and visual outputs, the controller scheduling onset of feedback cues such that actuation delays specific to each modality are compensated in advance, thereby ensuring that the wearer perceives multimodal corrective cues as simultaneous and coherent rather than temporally staggered.

17. The system of claim 1, wherein the microcontroller implements a long-term adaptation module that periodically analyzes stored rehabilitation session data to evaluate progressive changes in gait deviation magnitudes and muscle activation latencies, the module adjusting therapeutic thresholds by incrementally narrowing permissible tolerance ranges once sustained improvement is observed, thereby providing finer resolution corrective cues as the wearer advances through successive stages of rehabilitation.
